(19) United States
(12) Patent Application Publication
(10) Pub. No.: US 2024/0016874 A1
(43) Pub. Date: Jan. 18, 2024

(54) COMPOSITIONS AND METHODS FOR PREVENTING AND TREATING CORONAVIRUSES

(71) Applicant: NOVATEK THERAPEUTICS, LLC, Pearland, TX (US)

(72) Inventors: Abdulhafez Selim, Oakton, VA (US); Ahmad Omar Kaseb, Pearland, TX (US)

(73) Assignee: NOVATEK THERAPEUTICS, LLC, Pearland, TX (US)

(21) Appl. No.: 18/034,878

(22) PCT Filed: Oct. 28, 2021
(86) PCT No.: PCT/US2021/057145

§ 371 Date: May 1, 2023

Related U.S. Application Data
(60) Provisional application No. 63/107,266, filed on Oct. 29, 2020.

(51) Int. Cl. A61K 36/71; A61K 31/122; A61K 31/5365; A61K 47/14; A61K 9/48; A61P 31/14

(52) U.S. Cl. CPC A61K 36/71; A61K 31/122; A61K 31/5365; A61K 47/14; A61K 9/4858; A61P 31/14

(57) ABSTRACT

Method of preventing or treating coronaviruses, such as COVID-19 infection, in a subject, comprising administering to the subject an effective amount of thymoquinone, such as an effective amount of black seed oil.

### FIELD OF THE INVENTION

The present invention relates to compositions (e.g., compositions extracted from Nigella sativa seeds) and methods for preventing and treating coronaviruses (e.g., COVID-19).

### BACKGROUND

SARS-CoV-2 (“COVID-19”) was first identified as an illness in humans in December 2019 in Wuhan, China, which has since led to a global pandemic that has infected over 35 million people. It is mostly thought to be transmitted through contaminated air droplets of an infected individual, although how the virus is spread is still not entirely understood and currently being studied. While the most common symptoms of the disease are fever, cough, and lethargy, the virus can lead to permanent lung scaring, respiratory failure, and death in the most serious cases. For more mild infections, treatment is usually limited to rest at home while the virus runs its course. However, for more serious instances, current treatments include dexamethasone, remdesivir, and monoclonal antibodies, although no medication has been officially approved for treatment of SARS-CoV-2. Accordingly, there remains a need for effective therapies to treat COVID-19.

Six distinct strains of Human coronaviruses (HCoVs) have been described, in addition to the newly emerged COVID-19. Coronaviruses are enveloped, positive-sense, single-stranded RNA viruses of ˜30 kb. They infect a broad array of host species. They are essentially categorized into four genera; α, β, γ, and δ based on their genomic structure. α and β coronaviruses infect only mammals. Human coronaviruses such as 229E and NL63 are responsible for common cold and croup and belong to α coronavirus. In contrast, SARS-CoV, OC43, Middle East respiratory syndrome coronavirus (MERS-CoV) and SARS-CoV-2 are classified as β coronaviruses. SARS and MERS HCoV are the most aggressive strains of coronaviruses, leaving about 800 deaths each. SARS HCoV has a 10% mortality rate, while MERS HCoV has a 36% mortality rate, according to the WHO.

Human coronavirus 229E, also known as HCoV-229E, is a type of coronavirus that was first identified in the 1960s. It primarily infects human beings, although it has been found to infect bats as well. It is thought to be one of the most common viruses that cause the common cold, second only to rhinoviruses. HCoV-229E is transmitted through both droplet-respiration (i.e., through the droplets in the sneeze or cough of an infected person) and through physical touch of an infected person or surface. The virus is almost only ever active in the months of December through April, and has rarely been found in summer months. As cases tend to be more mild, symptoms tend to present as similar to those of an average cold, the most common ones being a sore throat, runny nose, and fever. However, more serious cases have been known to occur, primarily in immunocompromised patients, which can lead to lower respiratory tract infections such as pneumonia and bronchitis and can become life-threatening. Although there are no specific medications or treatments available for HCoV-229E, fever and pain medications, along with proper hydration, have been found to assist in relieving the symptoms as the virus runs its course.

Human coronavirus NL63 (“HCoV-NL63”) is a newer strand of coronavirus to infect humans. The first known case was found in a seven-month-old infant in Holland in 2004, though it has been known to exist in bats and palm civets for far longer. While it is not entirely known how the virus is transmitted, touching an infected person or surface is thought to play a key role, along with droplets from an infected person. Because HCov-NL63 is found primarily in children, the elderly, and immunocompromised patients, cases tend to be more serious, with common symptoms being upper respiratory tract infections, croup, and bronchitis for many of those infected. However, in those patients who are healthier and without any secondary health risks, cases tend to be far more mild, leading to less-severe symptoms of a sore throat and cough. Treatment for HCov-NL63 varies depending on the infection. In mild to moderate cases, the virus tends to go away on its own and relief can be found in pain and fever medication. In more severe cases, antivirals are often used as treatment, as well as the use of intravenous immunoglobin as an inhibitor, though no treatment has been officially approved by the FDA.

Human coronavirus HKU1 (“HCoV-HKU1) is one of the more recently identified coronavirus strands found to infect humans. It was first discovered in 2004, in a man from Hong Kong, although it is believed to have originated in rodents far earlier. Like many of the human coronavirus strands, symptoms are usually relatively mild, most commonly presenting similarly to the common cold; sore throat, coughing, and headache are among the most common symptoms. However, in more serious cases such as those that occur in those with weakened immune systems, symptoms can advance to bronchitis and pneumonia and can be life-threatening. The virus most often occurs in the later winter months and is most frequently found in children. It is theorized that it is transmitted through the respiratory droplets of an infected person's cough or sneeze, although transmission of HCoV-HKU1 is not entirely understood. While there is no officially approved vaccine or treatment for HCoV-HKU1. Accordingly, there remains a need for effective therapies to treat these other forms of coronavirus.

Nigella sativa, a dicotyledon of the Ranunculaceae family, is a bushy plant with white or blue flowers native to southern Europe, northern Africa and Asia Minor. Fruit capsules from N. sativa plants include white trigonal seeds that, once exposed to air, turn black. Extract oil obtained from the seeds of N. sativa, known as black seed, black cumin or Habatul-Bsarakah, have been employed in the Middle East and Asia, and contains the active ingredient thymoquinone, among other therapeutically components (e.g., structural derivatives of thymoquinone).

Thymoquinone can also be synthesized directly, and is publicly available. See, e.g., Chinese Patent Publication No. 103288618A; Catalogue Nos. 03416, Millipore Sigma, analytical standard grade.

### BRIEF SUMMARY OF THE INVENTION

One embodiment of the presently disclosed subject matter provides a method of preventing or treating COVID-19 infection in a subject comprising administering to the subject an effective amount of thymoquinone, such as, but not limited to, a composition that includes black seed oil. In certain embodiments, the thymoquinone can be administered prophylactically prior to a diagnosed infection to prevent COVID-19 infection, or alternatively can be administered subsequent to a diagnosed (or presumed) infection to treat COVID-19. In certain embodiments, an additional amount of thymoquinone can be administered along with the black seed oil. Still further, other embodiments of the presently disclosed subject matter include further administering an antiretroviral, such as dolutegravir, in combination with the thymoquinone (e.g., black seed oil).

In other embodiments, the presently disclosed subject matter provides a method of preventing or treating a coronavirus. For example, the coronavirus can be selected from SARS-CoV (“SARS”), SARS-CoV-2 (“COVID-19”), MERS-CoV, 229E (alpha coronavirus), NL63 (alpha coronavirus), (beta coronavirus), and HKU1 (beta coronavirus).

Other embodiments of the presently disclosed subject matter relate to use of composition comprising an effective amount of thymoquinone to treat a coronavirus, such as COVID-19, or a medicament labeled for use in treating a coronavirus, such as COVID-19.

The invention is further directed to the general and specific embodiments defined, respectively, by the claims appended hereto, which are incorporated by reference herein.

### BRIEF DESCRIPTION OF THE DRAWINGS

The following detailed description of exemplary embodiments of the subject disclosure will be better understood when read in conjunction with the appended drawings. For the purpose of illustrating the present disclosure, exemplary embodiments and data from exemplary embodiments are shown in the drawings. It should be understood, however, that the subject application is not limited to the precise arrangements and instrumentalities shown.

FIG. 1 is a plasmid map of the SARS-CoV-2-Spike-ΔC19 described in Example 2.

FIG. 2 depicts the protein sequence of SARS-CoV-2-614G-Spike-ΔC19 described in Example 2.

FIG. 3 depicts the protein sequence of SARS-CoV-2-UK variant Spike-ΔC19 described in Example 2.

FIG. 4 depicts the protein sequence of SARS-CoV-2-Delta variant Spike-ΔC19 described in Example 2.

FIG. 5 depicts the protein sequence of the protein sequence of SARS-CoV-2-Brazil variant Spike-ΔC19 described in Example 2.

FIG. 6 depicts the protein sequence of the Expi-293F-ACE2 stable cell line used for quality control, as described in Example 2.

FIG. 7 depicts a plot of the relative luminescence unity (RLU) for the 4 strains: 614G, Delta, UK, and Brazil against various concentrations of black seed oil, reflecting the luciferase activity and viral infectivity, as described in Example 2, and using a Firefly Luciferase Assay Kit.

FIG. 8 depicts a plot of the relative luminescence unity (RLU) for the 4 strains: 614G, Delta, UK, and Brazil against various concentrations of thymoquinone, reflecting the luciferase activity and viral infectivity, as described in Example 2, and using a Firefly Luciferase Assay Kit.

FIG. 9 depicts a plot of the relative luminescence unity (RLU) for the 4 strains: 614G, Delta, UK, and Brazil against various concentrations of oleic acid, reflecting the luciferase activity and viral infectivity, as described in Example 2, and using a Firefly Luciferase Assay Kit.

FIG. 10 depicts a plot of the relative luminescence unity (RLU) for the 4 strains: 614G, Delta, UK, and Brazil against various concentrations of linoleic acid, reflecting the luciferase activity and viral infectivity, as described in Example 2, and using a Firefly Luciferase Assay Kit.

FIG. 11 depicts a plot of the relative luminescence unity (RLU) for the 4 strains: 614G, Delta, UK, and Brazil against various concentrations of palmitic acid, reflecting the luciferase activity and viral infectivity, as described in Example 2, and using a Firefly Luciferase Assay Kit.

FIG. 12 depicts a plot of the relative luminescence unity (RLU) for the 4 strains: 614G, Delta, UK, and Brazil against various concentrations of oleic acid in the presence of thymoquinone, reflecting the luciferase activity and viral infectivity, as described in Example 2, and using a Firefly Luciferase Assay Kit.

FIG. 13 depicts a plot of the relative luminescence unity (RLU) for the 4 strains: 614G, Delta, UK, and Brazil against various concentrations of linoleic acid in the presence of thymoquinone, reflecting the luciferase activity and viral infectivity, as described in Example 2, and using a Firefly Luciferase Assay Kit.

FIG. 14 depicts a plot of the relative luminescence unity (RLU) for the 4 strains: 614G, Delta, UK, and Brazil against various concentrations of palmitic acid in the presence of thymoquinone, reflecting the luciferase activity and viral infectivity, as described in Example 2, and using a Firefly Luciferase Assay Kit.

FIG. 15 depicts a plot of the relative luminescence unity (RLU) for the 4 strains: 614G, Delta, UK, and Brazil against various concentrations of linoleic acid in the presence of oleic acid, reflecting the luciferase activity and viral infectivity, as described in Example 2, and using a Firefly Luciferase Assay Kit.

FIG. 16 depicts a plot of the relative luminescence unity (RLU) for the 4 strains: 614G, Delta, UK, and Brazil against various concentrations of palmitic acid in the presence of oleic acid, reflecting the luciferase activity and viral infectivity, as described in Example 2, and using a Firefly Luciferase Assay Kit.

FIG. 17 depicts a plot of the relative luminescence unity (RLU) for the 4 strains: 614G, Delta, UK, and Brazil against various concentrations of palmitic acid in the presence of linoleic acid, reflecting the luciferase activity and viral infectivity, as described in Example 2, and using a Firefly Luciferase Assay Kit.

FIG. 18 depicts a plot of the relative luminescence unity (RLU) against various concentrations of black seed oil, reflecting the luciferase activity and viral infectivity, as described in Example 3, and using a Firefly Luciferase Assay Kit.

FIG. 19 depicts a plot of the relative luminescence unity (RLU) against various concentrations of black seed oil in the presence of thymoquinone, reflecting the luciferase activity and viral infectivity, as described in Example 3, and using a Firefly Luciferase Assay Kit.

FIG. 20 depicts a plot of the relative luminescence unity (RLU) against various concentrations of thymoquinone, reflecting the luciferase activity and viral infectivity, as described in Example 3, and using a Firefly Luciferase Assay Kit.

FIG. 21 depicts a plot of the relative luminescence unity (RLU) against various concentrations of thymoquinone in the presence of black seed oil, reflecting the luciferase activity and viral infectivity, as described in Example 3, and using a Firefly Luciferase Assay Kit.

FIG. 22 depicts a plot of the relative luminescence unity (RLU) of various concentrations of black seed oil, reflecting the luciferase activity and the cell number, as described in Example 3, and using a Codex EnerCount cell growth Assay Kit.

FIG. 23 depicts a plot of the relative luminescence unity (RLU) of various concentrations of black seed oil in the presence of thymoquinone, reflecting the luciferase activity and the cell number, as described in Example 3, and using a Codex EnerCount cell growth Assay Kit.

FIG. 24 depicts a plot of the relative luminescence unity (RLU) of various concentrations of thymoquinone, reflecting the luciferase activity and cell number, as described in Example 3, and using a Codex EnerCount cell growth Assay Kit.

FIG. 25 depicts a plot of the relative luminescence unity (RLU) of various concentrations of thymoquinone in the presence of black seed oil, reflecting the luciferase activity and cell number, as described in Example 3, and using a Codex EnerCount cell growth Assay Kit.

FIG. 26 depicts a plot of the relative luminescence unity (RLU) of various concentrations of temsavir, temsavir+black seed oil, and temsavir+thymoquinone, reflecting the luciferase activity and viral infectivity, as described in Example 4, and using a Firefly Luciferase Assay Kit, the temsavir alone group shown as the top line.

FIG. 27 depicts a plot of the relative luminescence unity (RLU) of various concentrations of dolutegravir, dolutegravir+black seed oil, and dolutegravir+thymoquinone, reflecting the luciferase activity and viral infectivity, as described in Example 4, and using a Firefly Luciferase Assay Kit, the dolutegravir+1 μM Thymoquinone group shown as the top line.

FIG. 28 depicts a plot of the relative luminescence unity (RLU) of various lower concentrations of dolutegravir, dolutegravir+black seed oil, and dolutegravir+thymoquinone, reflecting the luciferase activity and viral infectivity, as described in Example 4, and using a Firefly Luciferase Assay Kit, the dolutegravir along group shown as the top line.

FIG. 29 depicts a model-based change of total symptoms burden by Study Arm in cohort 1 as described in Example 5, in which the line representing blackseed oil is non-linear and the line representing the placebo is more linear.

FIG. 30 depicts a model-based change of total symptoms burden by Study Arm in cohort 2 as described in Example 5, in which the line representing blackseed oil is non-linear and the line representing the placebo is more linear.

FIG. 31 depicts a model-based change of total symptoms burden by Study Arm in cohort 3 as described in Example 5, in which the line representing blackseed oil is non-linear and the line representing the placebo is more linear.

FIG. 32 depicts a comparison of % CD45RA+CCR7+(% CD4T)(% of CD4 Tcell) between the treatment arms (blackseed oil and placebo) on day 14 in cohort 1, the black seed arm shown on the left and the placebo arm shown on the right.

FIG. 33 depicts a comparison of % CD45RA+CCR7+(% CD8 T)(% of CD8 T cell) between the treatment arms (blackseed oil and placebo) on day 14 in cohort 1, the black seed arm shown on the left and the placebo arm shown on the right.

FIG. 34 depicts a comparison of CD45RA+CCR7+CD8T(abs.)(/μL) between the treatment arms (blackseed oil and placebo) on day 14 in cohort 1, the black seed arm shown on the left and the placebo arm shown on the right.

### DETAILED DESCRIPTION OF THE INVENTION

The invention can be more fully appreciated by reference to the following description, including the examples. Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. In addition, the materials, methods, and examples are illustrative only and not intended to be limiting.

For the sake of brevity, all publications, including patent applications, patents, and other citations mentioned herein, are incorporated by reference in their entirety. Citation of any such publication, however, shall not be construed as an admission that it is prior art to the present invention.

As used herein, the term “about” or “approximately” means within an acceptable range for a particular value as determined by one skilled in the art, and may depend in part on how the value is measured or determined, e.g., the limitations of the measurement system or technique. For example, “about” can mean a range of up to 20%, up to 10%, up to 5%, or up to 1% or less on either side of a given value. Alternatively, with respect to biological systems or processes, the term “about” can mean within an order of magnitude, within 5-fold, or within 2-fold on either side of a value. Numerical quantities given herein are approximate unless stated otherwise, meaning that the term “about” or “approximately” can be inferred when not expressly stated.

To provide a more concise description, some of the quantitative expressions given herein are not qualified with the term “about.” It is understood that, whether the term “about” is used explicitly or not, every quantity given herein is meant to refer to both the actual given value and the approximation of such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity for which that could be obtained under the particular stoichiometric conditions. Concentrations that are given as percentages refer to mass ratios, unless indicated differently.

As used herein, the terms “a,” “an,” and “the” are to be understood as meaning both singular and plural, unless explicitly stated otherwise. Thus, “a,” “an,” and “the” (and grammatical variations thereof where appropriate) refer to one or more.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof, unless limitation to the singular is explicitly stated.

The terms “comprising” and “including” are used herein in their open, non-limiting sense. Other terms and phrases used in this document, and variations thereof, unless otherwise expressly stated, should be construed as open ended, as opposed to limiting. Thus, the term “example” is used to provide exemplary instances of the item in discussion, not an exhaustive or limiting list thereof. Similarly, adjectives such as “conventional,” “traditional,” “normal,” “criterion,” “known,” and terms of similar meaning should not be construed as limiting the item described to a given time period or to an item available as of a given time, but they should be read to encompass conventional, traditional, normal, or criterion technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. As will become apparent to one of ordinary skill in the art after reading this document, the illustrated embodiments and their various alternatives may be implemented without confinement to the illustrated examples.

The term “composition” is intended to encompass a product including the herein described extracts and the inert ingredient(s) (pharmaceutically acceptable excipients) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. In certain embodiments, a “composition,” as used herein, is pharmaceutically acceptable and suitable for oral administration. In alternative embodiments, a “composition,” as used herein, is pharmaceutically acceptable and suitable for topical administration.

The term “carrier” refers to an adjuvant, vehicle, or excipients, with which the compound is administered. In certain embodiments of this invention, the carrier is a solid carrier. Suitable pharmaceutical carriers include those described in Remington: The Science and Practice of Pharmacy, 21^{st} Ed., Lippincott Williams & Wilkins (2005).

The term “dosage form,” as used herein, is the form in which the dose is to be administered to the subject or patient. The active extract can be administered as part of a formulation that includes nonmedical agents. The dosage form has unique physical and pharmaceutical characteristics. Dosage forms, for example, can be solid, liquid, gel or gaseous. “Dosage forms” can include for example, a capsule, tablet, caplet, gel caplet (gelcap), syrup, a powder or spray for buccal or intranasal administration, a chewable form, and an oral liquid solution. In a specific embodiment, the dosage form is a solid dosage form, and more specifically, comprises a tablet or capsule.

The term “pharmaceutically acceptable,” as used in connection with compositions of the invention, refers to molecular entities and other ingredients of such compositions that are physiologically tolerable and do not typically produce untoward reactions when administered to an animal (e.g., human) according to their intended mode of administration (e.g., oral or parenteral).

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluents to facilitate administration of an agent and that is compatible therewith. Examples of excipients include calcium carbonate, calcium phosphate, various sugars and types of starch, cellulose derivatives, gelatin, vegetable oils, and polyethylene glycols. Suitable pharmaceutical carriers include those described in Remington: The Science and Practice of Pharmacy, 21^{st} Ed., Lippincott Williams & Wilkins (2005).

As used herein, the term “inert” refer to any inactive ingredient of a described composition. The definition of “inactive ingredient” as used herein follows that of the U.S. Food and Drug Administration, as defined in 21 C.F.R. 201.3(b)(8), which is any component of a drug product other than the active ingredient.

As used herein, “suitable for oral administration” or “suitable for topical administration” refers to a sterile, pharmaceutical product produced under good manufacturing practices (GMP). The term “suitable for oral administration” or “suitable for topical administration” can, when specified, also mean approved by a regulatory agency of the Federal or a state government or listed in the U.S. Pharmacopeia or other generally recognized pharmacopeia for use in animals (e.g. mammals), and more particularly in humans.

As used herein, the term “disorder” is used interchangeably with “disease” or “condition”. For example, a neurological disorder also means a neurological disease or a neurological condition.

The terms “treat,” “treating,” and “treatment” cover therapeutic methods directed to a disease-state in a subject and include: (i) preventing the disease-state from occurring, in particular, when the subject is predisposed to the disease-state but has not yet been diagnosed as having it; (ii) inhibiting the disease-state, e.g., arresting its development (progression) or delaying its onset; and (iii) relieving the disease-state, e.g., causing regression of the disease state until a desired endpoint is reached. These terms also include ameliorating a symptom of a disease (e.g., reducing the pain, discomfort, or deficit), wherein such amelioration may be directly affecting the disease (e.g., affecting the disease's cause, transmission, or expression) or not directly affecting the disease.

As used in the present disclosure, the term “effective amount” is interchangeable with “therapeutically effective amount” and means an amount or dose of a compound or composition effective in treating the particular disease, condition, or disorder disclosed herein, and thus “treating” includes producing a desired preventative, inhibitory, relieving, or ameliorative effect. In methods of treatment according to the invention, “an effective amount” of at least one compound is administered to a subject (e.g., a mammal). The “effective amount” will vary, depending on the compound, the disease (and its severity), the treatment desired, age and weight of the subject, etc.

As used herein, the phrase “in combination” refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents remain in the subject's body, they are considered to be administered “in combination”. As will be explained below, for example, black seed oil (or thymoquinone) can be administered in combination with dolutegravir.

As used herein, the term “modulate” refers to change in a parameter (e.g., a change in a binding interaction or an activity, etc.). Modulation can refer to an increase or a decrease in the parameter (e.g., an increase or decrease in binding, an increase or decrease in activity, etc.).

The terms “individual,” “subject,” and “patient” are used interchangeably herein and can be a vertebrate, in particular, a mammal, more particularly, a primate (including non-human primates and humans) and include a laboratory animal in the context of a clinical trial or screening or activity experiment. Thus, as can be readily understood by one of ordinary skill in the art, the compositions and methods of the present invention are particularly suited to administration to any vertebrate, particularly a mammal, and more particularly, a human.

As used herein, the term “black seed oil” or “blackseed oil” shall refer to compositions (e.g., extracts) obtained from Nigella sativa seeds.

As used herein, the term “COVID-19” includes all variants of COVID-19.

A composition of the present invention, alone or in combination with other active ingredients, can be administered to a subject in a single dose or multiple doses over a period of time, generally by oral or parenteral administration. As used herein, the terms “therapeutically effective amount,” refers to the amount of the composition of the invention that results in a therapeutic or beneficial effect, following its administration to a subject.

The concentration of the substance is selected so as to exert its therapeutic effect, but low enough to avoid significant side effects within the scope and sound judgment of the skilled artisan. The effective amount of the composition may vary with the age and physical condition of the biological subject being treated, the severity of the condition (e.g., severity of COVID-19 symptoms), the duration of the treatment, the nature of concurrent therapy, the specific compound, composition or other active ingredient employed, the particular carrier utilized, and like factors.

Those of skill in the art can readily evaluate such factors and, based on this information, determine the particular effective concentration and unit dosage amount of a composition of the present invention to be used for an intended purpose. For example, suitable dosage amounts can be obtained by extrapolating dose-response curves derived from in vitro or animal model test systems. See, for example, Goodman and Gilman's The Pharmacological Basis of Therapeutics, Joel G. Harman, Lee E. Limbird, Eds.; McGraw Hill, New York, 2001; The Physician's Desk Reference, Medical Economics Company, Inc., Oradell, N.J., 1995; and Drug Facts and Comparisons, Facts and Comparisons, Inc., St. Louis, Mo., 1993). The precise dose to be employed in the formulation will also depend on the route of administration, and the seriousness of the viral infection, and can be decided according to the judgment of the practitioner and each patient's circumstances. Various administration patterns will be apparent to those skilled in the art.

The dosage ranges for the administration of the compositions of the present invention are those large enough to produce the desired therapeutic effect.

Those skilled in the art will recognize that initial indications of the appropriate therapeutic dosage of the compositions of the invention can be determined in in vitro and in vivo animal model systems, and in human clinical trials. One of skill in the art would know to use animal studies and human experience to identify a dosage that can safely be administered without generating toxicity or other side effects. In certain circumstances, it is preferred that the therapeutic dosage be close to the maximum tolerated dose. For chronic preventive use, relatively lower dosages can be desirable.

In certain non-limiting, exemplary embodiments from about 100 mg to about 15 g, or from about 200 mg to about 7000 mg, or from about 300 mg to about 5000 mg, or from about 500 mg to about 5000 mg of black seed oil (e.g., 3 g) of black seed oil can be administered to a subject (e.g., human) per day to prophylactically prevent or treat an existing coronavirus infection (e.g., COVID-19). These amounts can be administered once daily or over several doses equally spaced throughout the day. Dosing amounts are based on black seed oil having a concentration of thymoquinone of about 1.5-2.5 wt %; dosing amounts to be adjusted higher or lower depending on the amount of thymoquinone present in the black seed oil.

Relatively lower amounts can be administered when used prophylactically. In certain embodiments in which the black seed oil is used to prevent a coronavirus infection, from about 250 mg to about 3000 mg, or from 500 mg to about 1500 mg, or from about 750 mg to about 1250 mg (e.g. 1000 mg) can be administered to a subject (e.g., human) per day to prevent a coronavirus infection (e.g., COVID-19).

In certain embodiments in which the black seed oil is used to treat an existing coronavirus infection, from about 500 mg to about 10 g, or from 1000 mg to about 8000 mg, or from about or from about 2000 mg to about 4000 mg (e.g. 3000 mg) can be administered to a subject (e.g., human) per day to treat an existing coronavirus infection (e.g., COVID-19).

In certain non-limiting, exemplary embodiments from about 0.5 mg to about 1000 mg, or from about 1 mg to about 500 mg, or from about 5 mg to about 300 mg, or from about 10 mg to about 150 mg of thymoquinone can be administered to a subject (e.g., human) per day to prophylactically prevent or treat an existing coronavirus infection (e.g., COVID-19). These amounts can be administered once daily or over several doses equally spaced throughout the day.

Relatively lower amounts can be administered when used prophylactically. In certain embodiments in which the thymoquinone is used to prevent a coronavirus infection, from about 5 mg to about 80 mg, or from 10 mg to about 40 mg, or from about 20 mg to about 30 mg (e.g. 25 mg) can be administered to a subject (e.g., human) per day to prevent a coronavirus infection (e.g., COVID-19).

In certain embodiments in which the thymoquinone is used to treat an existing coronavirus infection, from about 25 mg to about 300 mg, or from 50 mg to about 150 mg, or from about or from about 75 mg to about 125 mg (e.g. 100 mg) can be administered to a subject (e.g., human) per day to treat an existing coronavirus infection (e.g., COVID-19).

In certain non-limiting, exemplary embodiments from about 0.25 mg to about 1 g, or from about 0.5 mg to about 500 mg, or from about 0.75 mg to about 250 mg, or from about 1 mg to about 150 mg of antiretroviral (e.g., dolutegravir) can be administered to a subject (e.g., human) per day to prophylactically prevent or treat an existing coronavirus infection (e.g., COVID-19). In certain embodiments, 10 mg, 25 mg or 50 mg of antiretroviral is administered from about once to about 10 times per day. These amounts can be administered once daily or over several doses equally spaced throughout the day.

Relatively lower amounts can be administered when used prophylactically. In certain embodiments in which an anti is used to prevent a coronavirus infection, from about 0.5 mg to about 100 mg, or from 1 mg to about 50 mg, or from about 10 mg to about 40 mg (e.g. 25 mg) of blackseed oil can be administered to a subject (e.g., human) per day to prevent a coronavirus infection (e.g., COVID-19).

In certain embodiments in which the antiretroviral is used, in combination, to treat an existing coronavirus infection, from about 5 mg to about 300 mg, or from 25 mg to about 150 mg can be administered to a subject (e.g., human) per day to treat an existing coronavirus infection (e.g., COVID-19).

The compounds and compositions of the present disclosure, e.g., black seed oil and/or antiretroviral, can be administered systemically, for example, by an oral or parenteral route of administration. Oral formulations of the present disclosure can be obtained commercially (e.g., black seed oil soft gel capsules) and/or formulated from available raw ingredients (e.g. formulated as tablets from sources of dolutegravir API).

In alternative embodiments, the compounds and compositions of the present disclosure can be administered via injection (e.g., intravenously, intramuscularly). Injectable formulations, composed of carriers and ingredients (e.g., water for injection) suitable for injection can be prepared by one of ordinary skill in the art. Similarly, compositions suitable for use as a suppository or as a nasal spray can be prepared by one of ordinary skill in the art, and administered in accordance with the presently disclosed subject matter.

For convenience, embodiments of the present invention are described in the context of treating or SARS-CoV-2, i.e., COVID-19. In alternative embodiments, the compounds and compositions of the present disclosure, e.g., a composition containing black seed oil, can be used to treat other coronaviruses, such as, but not limited to, SARS-CoV (“SARS”), MERS-CoV, 229E (alpha coronavirus), NL63 (alpha coronavirus), 0C43 (beta coronavirus), and HKU1 (beta coronavirus).

In one embodiment, the treatment of SARS-CoV (“SARS”) by administering a black seed oil and/or an amount of thymoquinone is excluded.

Black Seed Oil

The oil of black seeds contains thymoquinone (TQ), palmitic acid, linoleic acid, dithymoquinone, thymohydroquinone, thymol, carvacrol, nigellimine-N-oxide, nigellicine, nigellidine, and alpha-hederin. While thymoquinone is conventionally considered to be an active component, other components of black seed oil are also, according to non-binding theory, believed to impart a beneficial therapeutic effect.

In certain embodiments, the amount of thymoquinone present in the administered composition of black seed oil is at least 0.5 wt %, or at least 0.75 wt %, or at least lwt %, or at least 1.25 wt %, or at least 1.5 wt %, at least 1.6 wt %, or at least 1.75 wt %, at least 2 wt %, at least 2.1 wt %, or at least or 2.5 wt %, based on the total weight of the composition. As previously discussed, dosing amounts of the black seed oil can be adjusted based on the concentration of thymoquinone in the administered black seed oil composition.

In certain embodiments, the black seed oil is for oral administration and is formulated with an enteric coating by processing techniques known to those of ordinary skill in the art. Alternatively, or in addition, the formulation can be provided with taste-masking agents and other flavors to improve the taste of the black seed oil and improve patient compliance, particularly for prophylactic embodiments.

Antiretroviral Combination Therapy

In certain embodiments, an effective amount of antiretroviral (e.g., dolutegravir, or a pharmaceutically acceptable salt thereof) is administered to a subject to prevent or treat a coronavirus (e.g., COVID-19). The antiretroviral can be administered in combination with a source of thymoquinone (e.g., black seed oil).

Effective amounts of the antiretroviral (e.g., dolutegravir) can be determined by one of ordinary skill in the art. In exemplary embodiments, the amount of dolutegravir administered per day can range from about 1 mg to about 20 mg for prophylactic purposes and from about 25 mg to about 100 mg per day for treating an existing infection. Other amounts can be provided in alternative embodiments.

Structural or functional derivatives of dolutegravir can also be administered in combination with thymoquinone and/or black seed oil according to certain embodiments of the present invention to prevent or treat coronaviruses. For example, cabotegravir or pharmaceutically acceptable salts thereof, bictegravir or pharmaceutically acceptable salts thereof, can be administered according to the present disclosure.

Reference will now be made to the embodiments of the present invention, examples of which are illustrated by and described in conjunction with the accompanying examples. While certain embodiments are described herein, it is understood that the described embodiments are not intended to limit the scope of the invention. On the contrary, the present disclosure is intended to cover alternatives, modifications, and equivalents that can be included within the invention as defined by the appended claims.

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### EXAMPLES

### Example 1: Viral Entry: Molecular Docking Analysis

Viral entry of the COVID-19 virus occurs through the binding of viral spike proteins to the angiotensin-converting enzyme 2 (ACE2). See, e.g., Lukassen, S., Chua, R. L., Trefzer, T., Kahn, N. C., Schneider, M. A., Muley, T., . . . & Hennig, B. P. (2020). SARS-CoV-2 receptor ACE 2 and TMPRSS 2 are primarily expressed in bronchial transient secretory cells. The EMBO journal, 39(10), e105114.

Thymoquinone was docked with the viral-receptor complex (PDB: 3SCK). 3SCK complex has 4 chains: 2 chains (A, and B) of ACE2 receptors and 2 chains of the viral spike glycoprotein (E, and F). The resulting complex was analyzed for interface-interactions and then compared to the original complex.

Analysis using the computer-assisted, online molecular modelling tool mcule.com showed that thymoquinone caused significant disruption of viral spike-ACE2 receptor interaction: TQ caused reduced the number of hydrogen bonds from 37 to 24 (about 35.0% reduction); and reduced the number of salt bridges from 16 to 5 (about 69.0% reduction). Overall, interfacing bonds decreased by about of 44.0% as a result of TQ. These findings are significant and can provide a meaningful prophylactic and therapeutic effect.

Given the positive data analysis of thymoquinone, other key constituents of black seed oil were analyzed, i.e., palmitic acid, linoleic acid and palmitic acid. The analysis demonstrated similar outcomes with linoleic acid and palmitic acid.

This process was repeated to assess the interaction between dolutegravir and the viral-receptor complex (PDB: 3SCK). By comparing the viral protein-cell receptor complex (3 sck) before and after drug docking, the number of bonds decreased dramatically: Hydrogen bonds decreased from 37 to 24, and more importantly, the number of salt bridges decreased from 16 to 5. These data suggest that dolutegravir can have a meaningful disruption of the virus-receptor interactions, and can similarly provide a meaningful prophylactic and therapeutic effect.

### Example 2: In Vitro Viral Studies—Black Seed Oil, Thymoquinone and Fatty Acids

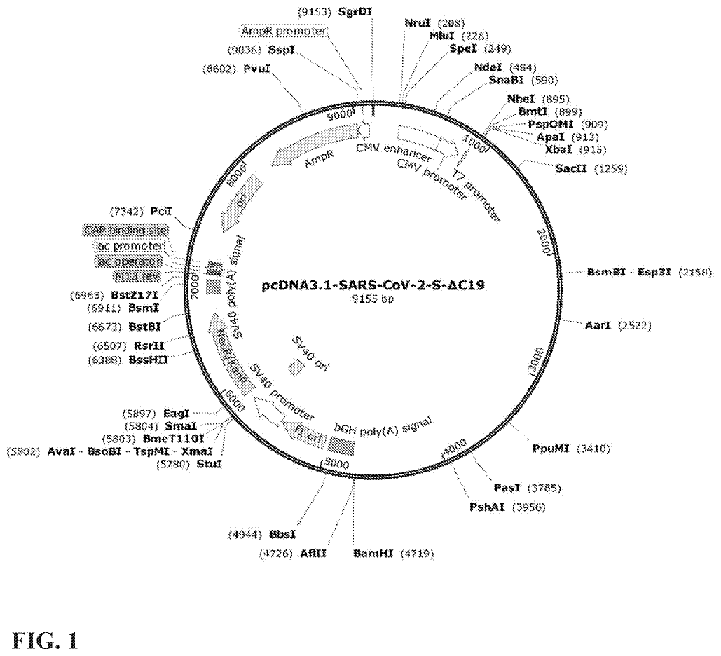

Murine Leukemia Virus (MLV) particles pseudotyped with a SARS-CoV-2 Spike protein construct were generated in HEK293T cells (Cat #CRL-3216, ATCC) following a protocol described for SARS-CoV, modified as described below. See Millet, J. K. & Whittaker, G. R. Murine Leukemia Virus (MLV)-based Coronavirus Spikepseudotyped Particle Production and Infection. Bio Protoc 6(2016); Chen, C. Z. et al. Identifying SARS-CoV-2 entry inhibitors through drug repurposing screens of SARS-S and MERS-S pseudotyped particles, bioRxiv (2020). All the plasmid DNAs were purified with ZymoPURE II Plasmid Midiprep Kit (Cat #D4201, Zymo Research). The plasmid map of SARS-CoV-2-Spike expression vector is depicted in FIG. 1.

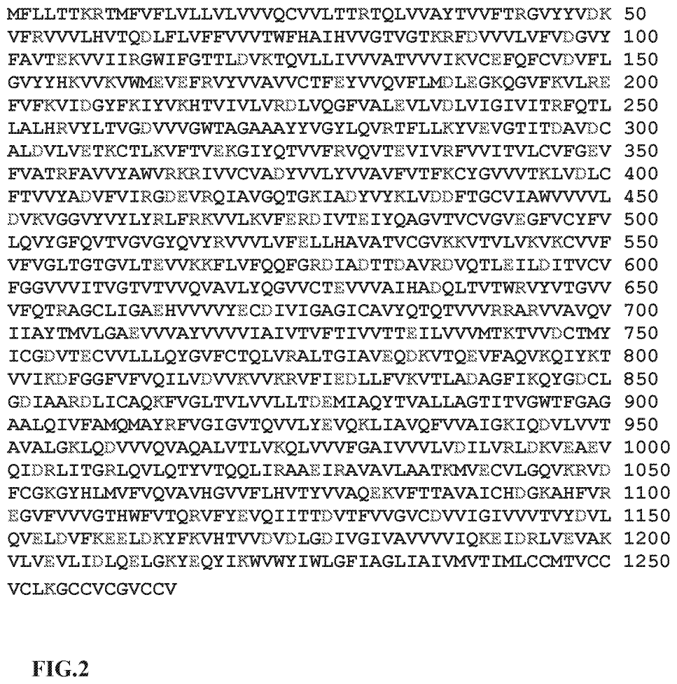

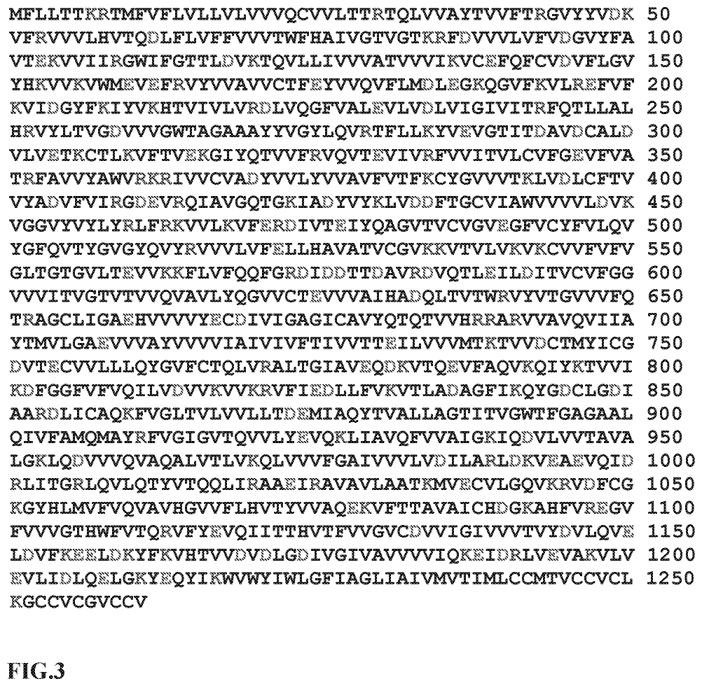

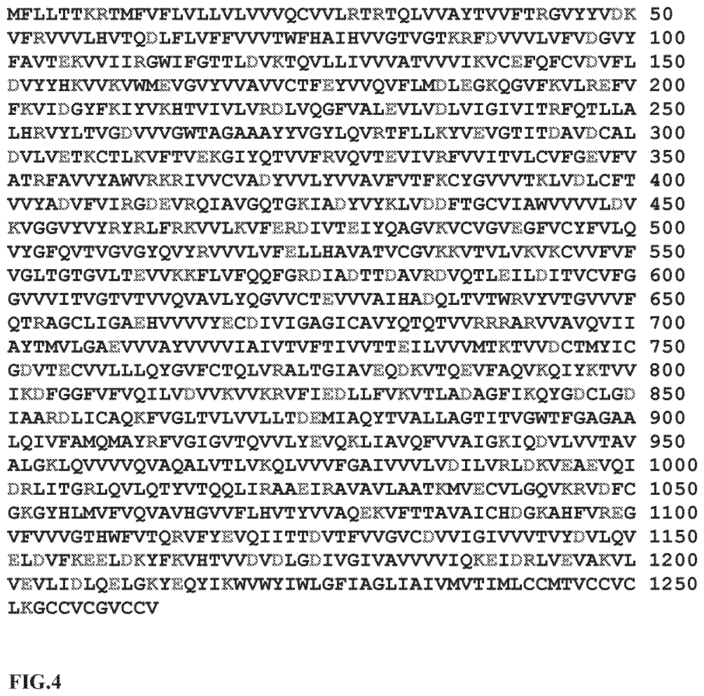

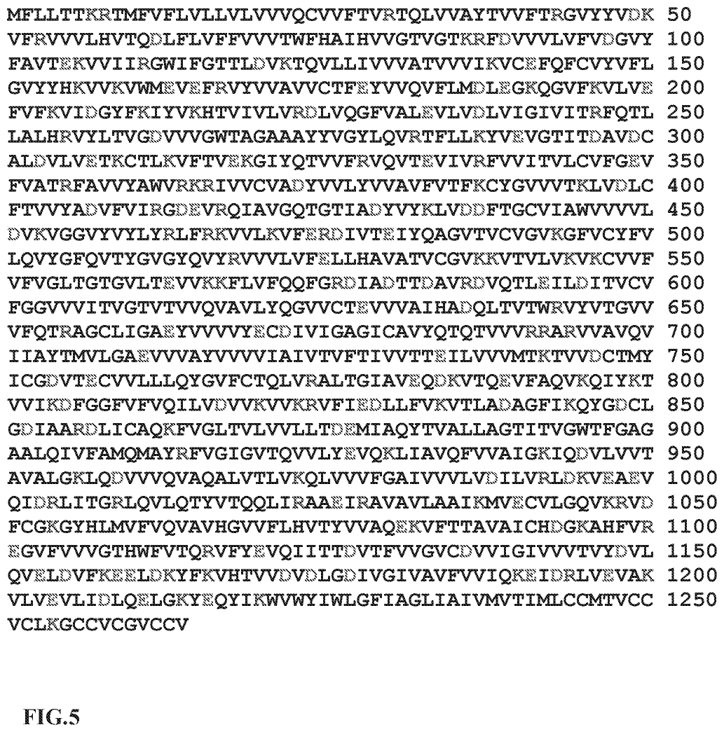

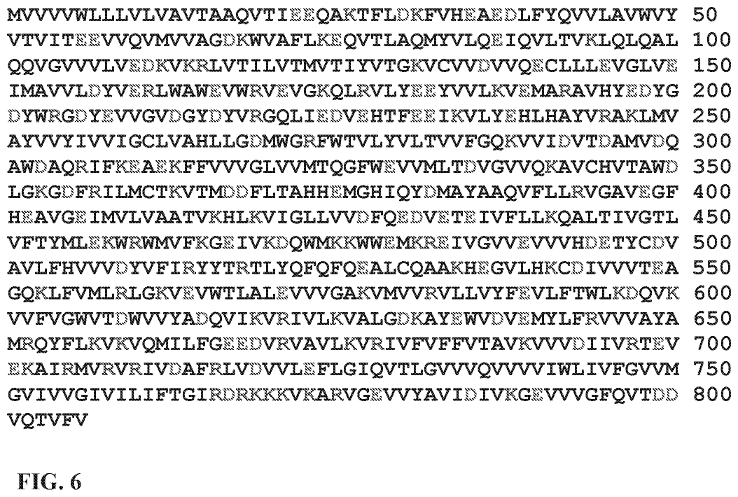

In particular, 8 million HEK293T cells were plated into a 10-cm tissue culture dish (Cat #sc-251460, Santa Cruz) in 16 ml DMEM (Cat #25-500, Genesee Scientific)+10% FBS (Cat #35-010-CV, Corning Life Sciences) without any antibiotics. On the 2nd day, the cells were transfected with 8 μg pTG-Luc, 6 μg pCMV-MLVgag-pol and 6 μg pcDNA3.1-SARS-CoV-2-Spike-ΔC19 of the variants specified in FIGS. 2-5 using Lipofectamine 3000 reagent (Cat #L3000015, ThermoFisher). The cells were cultured for an additional 48 hr. The supernatant was collected into a 50-ml Falcon tube and spun at 290×g for 7 min. The supernatant (pseudotyped virus solution) was then passed through a 0.45 lam filter (Cat #sc-358814, Santa Cruz) using appropriate syringe. The pseudotyped virus solution was then aliquoted into cryovials and stored at −80° C. Each 10-cm cell culture dish produces about 16 ml SARS-CoV-2-PP. The SARS-CoV-2-PP was tested for the quality control with the HEK293-ACE2 cell line (FIG. 6), created at Codex BioSolutions.

Black seed oil was obtained having a thymoquinone content of about 2 wt %. Thymoquinone (Cat #03416) was purchased from Millipore Sigma. Oleic acid (Cat #01008), Linoleic acid (Cat #L1376) and Palmitic acid (Cat #P0500) were purchased from MilliporeSigma.

The following regimens were tested:
1. Blackseed oil alone;
2. Thymoquinone alone;
3. Oleic acid alone;
4. Linoleic acid alone;
5. Palmitic acid alone.

The day before infection with the SARS-CoV-2-PP described above, 7.5K Expi-293F-ACE2 cells were plated into a 384-well white clear plate (Cat #353963, Corning Life Sciences) precoated with Poly D Lysine (Cat #3439-100-1, Trevigen, Inc) in 15 μl culture medium (DMEM+10% FetalClone II Serum, Cat #SH3006603, Fisher Scientific). The cell plate was placed in a CO₂ incubator and maintained at 37° C.

On the second day, the compositions to be tested were diluted in the culture medium on a 96-well compound plate as shown below in Tables 1-4, 5× of the final concentrations:

TABLE 1
An example of Black seed Oil concentrations in a compound dilution plate.

| Sample # | 1 | 2 | 3 | 4 | 5 | 6 | 7 | 8 | 9 | 10 | 11 | 12 |
|---|---|---|---|---|---|---|---|---|---|---|---|---|
| Black seed | 100% | 30% | 10% | 3% | 1% | 0.3% | 0.1% | 0.03% | 0.01% | 0.003% | 0.0001% | 0 |
| Oil | | | | | | | | | | | | |

TABLE 2
An example of Thymoquinone concentrations in a compound dilution plate.

| Sample # | 1 | 2 | 3 | 4 | 5 | 6 | 7 | 8 | 9 | 10 | 11 | 12 |
|---|---|---|---|---|---|---|---|---|---|---|---|---|
| Thymoquinone | 5000 | 1500 | 500 | 150 | 50 | 15 | 5 | 1.5 | 0.5 | 0.15 | 0.05 | 0 |
| (μM) | | | | | | | | | | | | |

TABLE 3
An example of Oleic acid concentrations in a compound dilution plate.

| Sample # | 1 | 2 | 3 | 4 | 5 | 6 | 7 | 8 | 9 | 10 | 11 | 12 |
|---|---|---|---|---|---|---|---|---|---|---|---|---|
| Oleic acid | 50 | 15 | 5 | 1.5 | 0.5 | 0.15 | .05 | .015 | .005 | .0015 | .0005 | 0 |
| (mg/ml) | | | | | | | | | | | | |

TABLE 4
An example of Linoleic acid concentrations in a compound dilution plate.

| Sample # | 1 | 2 | 3 | 4 | 5 | 6 | 7 | 8 | 9 | 10 | 11 | 12 |
|---|---|---|---|---|---|---|---|---|---|---|---|---|
| Linoleic acid | 50 | 15 | 5 | 1.5 | 0.5 | .15 | .05 | .015 | .005 | .0015 | .0005 | 0 |
| (mg/ml) | | | | | | | | | | | | |

TABLE 5
An example of Palmitic acid concentrations in a compound dilution plate.

| Sample # | 1 | 2 | 3 | 4 | 5 | 6 | 7 | 8 | 9 | 10 | 11 | 12 |
|---|---|---|---|---|---|---|---|---|---|---|---|---|
| Palmitic acid | 10 | 3 | 1 | 0.3 | 0.1 | .03 | .01 | .003 | .001 | .0003 | .0001 | 0 |
| (mg/ml) | | | | | | | | | | | | |

Eighty (80) μ1 of SARS-CoV-2 MLV psuedoparticles where mixed with 32 μl of each sample, prepared as described above, and incubated at 37° C. for 10 min. After the medium in each well of 384-well cell plate was removed, 17.5 μl of each compound-pp mixture was added into each well. Examples of final concentrations of black seed oil and thymoquinone on the 384-well assay plate are set forth below in Table 5:

TABLE 6
An example of final concentrations of Black seed
Oil and Thymoquinone on the 384-well assay plate.

| | 1 | 2 | 3 | 4 | 5 | 6 | 7 | 8 | 9 | 10 | |
|---|---|---|---|---|---|---|---|---|---|---|---|
| A | 20% | 20% | 6% | 6% | 2% | 2% | 1% | 1% | 0% | 0% | Black seed Oil |
| B | 20% | 20% | 6% | 6% | 2% | 2% | 1% | 1% | 0% | 0% | Black seed Oil |
| C | 1000 | 1000 | 300 | 300 | 100 | 100 | 30 | 30 | 10 | 10 | Thymoquinone (μM) |
| D | 1000 | 1000 | 300 | 300 | 100 | 100 | 30 | 30 | 10 | 10 | Thymoquinone (μM) |
| E | 10 | 10 | 3 | 3 | 1 | 1 | .3 | .3% | .1 | .1 | Oleic Acid (mg/ml) |
| F | 10 | 10 | 3 | 3 | 1 | 1 | .3 | .3% | .1 | .1 | Oleic Acid (mg/ml) |
| G | 10 | 10 | 3 | 3 | 1 | 1 | .3 | .3 | .1 | .1 | Linoleic Acid (mg/ml) |
| H | 10 | 10 | 3 | 3 | 1 | 1 | .3 | .3 | .1 | .1 | Linoleic Acid (mg/ml) |
| I | 2 | 2 | .6 | .6 | .2 | .2 | .06 | .06 | .02 | .02 | Palmitic Acid (mg/ml) |
| J | 2 | 2 | .6 | .6 | .2 | .2 | .06 | .06 | .02 | .02 | Palmitic Acid (mg/ml) |

| | 11 | 12 | 13 | 14 | 15 | 16 | 17 | 18 | 19 |
|---|---|---|---|---|---|---|---|---|---|
| A | 0.1% | 0.1% | 0.0% | 0.0% | 0.01% | 0.01% | 0.00% | 0.00% | 0.001% |
| B | 0.1% | 0.1% | 0.0% | 0.0% | 0.01% | 0.01% | 0.00% | 0.00% | 0.001% |
| C | 3 | 3 | 1 | 1 | .3 | .3 | .1 | .1 | .03 |
| D | 3 | 3 | 1 | 1 | .3 | .3 | .1 | .1 | .03 |
| E | .03 | .03 | .01 | .01 | .003 | .003 | .001 | .001 | .0003 |
| F | .03 | .03 | .01 | .01 | .003 | .003 | .001 | .001 | .0003 |
| G | .03 | .03 | .01 | .01 | .003 | .003 | .001 | .001 | .0003 |
| H | .03 | .03 | .01 | .01 | .003 | .003 | .001 | .001 | .0003 |
| I | .006 | .006 | .002 | .002 | .0006 | .0006 | .0002 | .0002 | .00006 |
| J | .006 | .006 | .002 | .002 | .0006 | .0006 | .0002 | .0002 | .00006 |

| | 20 | 21 | 22 | 23 | 24 |
|---|---|---|---|---|---|
| A | 0.001% | .0002% | .0002% | 0.0% | 0.0% |
| B | 0.001% | .0002% | .0002% | 0.0% | 0.0% |
| C | .03 | .01 | .01 | 0 | 0 |
| D | .03 | .01 | .01 | 0 | 0 |
| E | .0003 | .0001 | .0001 | 0 | 0 |
| F | .0003 | .0001 | .0001 | 0 | 0 |
| G | .0003 | .0001 | .0001 | 0 | 0 |
| H | .0003 | .0001 | .0001 | 0 | 0 |
| I | .00006 | .00002 | .00002 | 0 | 0 |
| J | .00006 | .00002 | .00002 | 0 | 0 |

The plate was centrifuged at 54 g for 15 min at 4° C. and additional 7.5 μl of the culture medium was then added into each well. The total final volume in each well was 25 μl. The cells were then incubated at 37° C. for 42 hr. Luciferase activities were measured with Firefly Luciferase Assay Kit (CB-80552-010, Codex BioSolutions Inc). IC50 values were calculated based on curve fitting in GraphPad Prism.

At the same time, the cell toxicities (Tox) of the fatty acids on HEK293-ACE2 cells was tested using Codex's EnerCount cell growth assay kit which measures the ATP levels inside the cells (Cat #CB-80551-010, Codex BioSolutions).

The day before the assay, 7.5K HEK293-ACE2 cells were plated into each well of a 384-well white clear plate (Cat #353963, Corning Life Sciences) precoated with Poly D Lysine (Cat #3439-100-1, Trevigen, Inc) in 15 id culture medium (DMEM+10% FetalClone II Serum, Cat #SH3006603, Fisher Scientific). The cell plate was placed in a CO₂ incubator (37° C.). On the second day, the compounds to be tested were diluted in the culture medium on a 96-well compound plate shown above in Tables 3-5, shown at 5× of the final concentrations.

The plate was taken out of the incubator and the medium in each well was removed. 20 μl culture medium (DMEM+10% FetalClone II Serum, Cat #SH3006603, Fisher Scientific) was added back into each well. 5 μl of the compound (5×) prepared as shown in Table 3-5 was added into each well. The final concentration is the same as in Table 6, Row E-J. The plate was centrifuged at 54 g for min at 4° C. The cells were then incubated at 37° C. for 42 hr. Luciferase activities were measured with Codex's EnerCount cell growth assay kit.

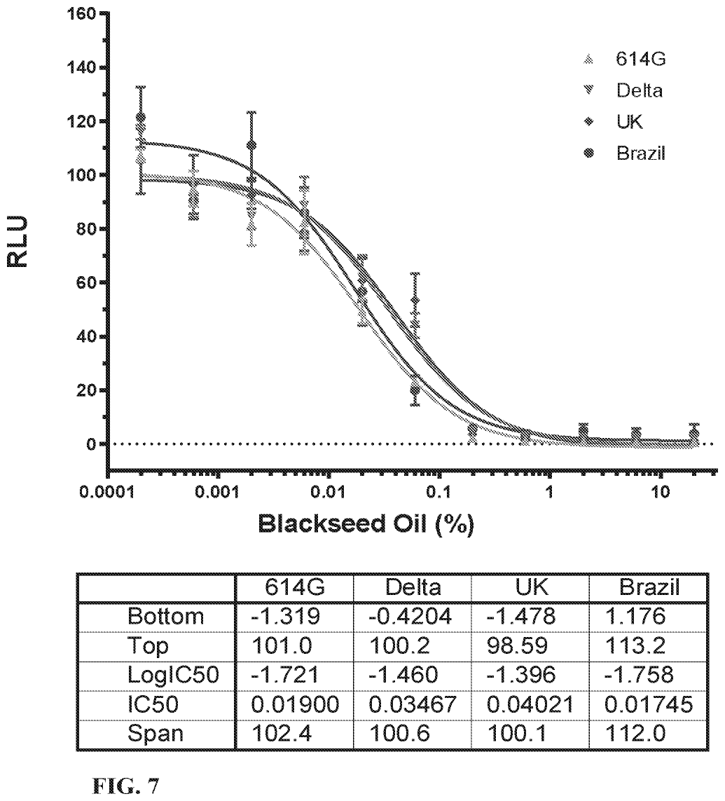

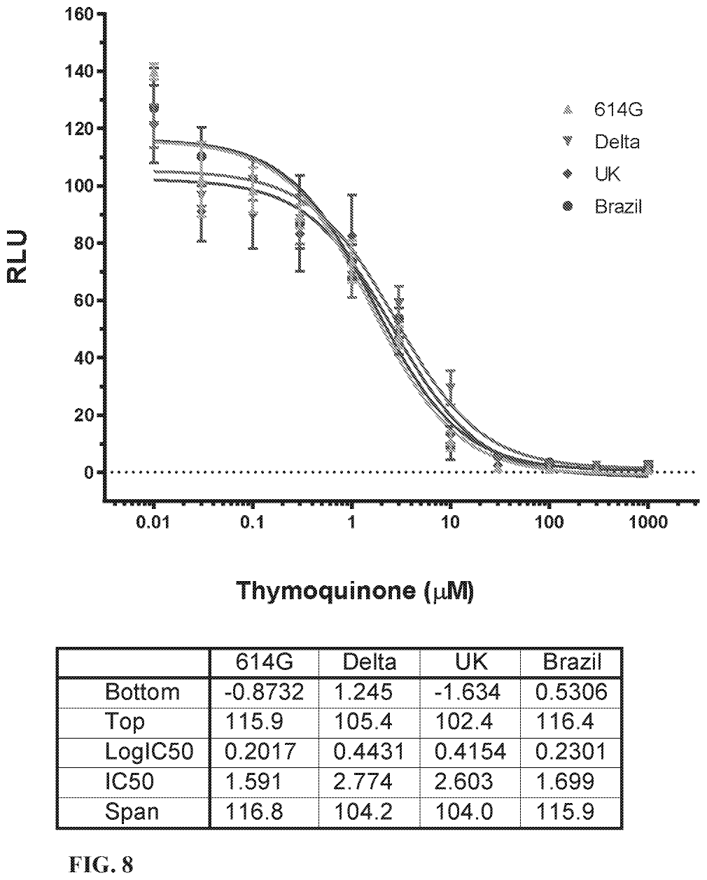

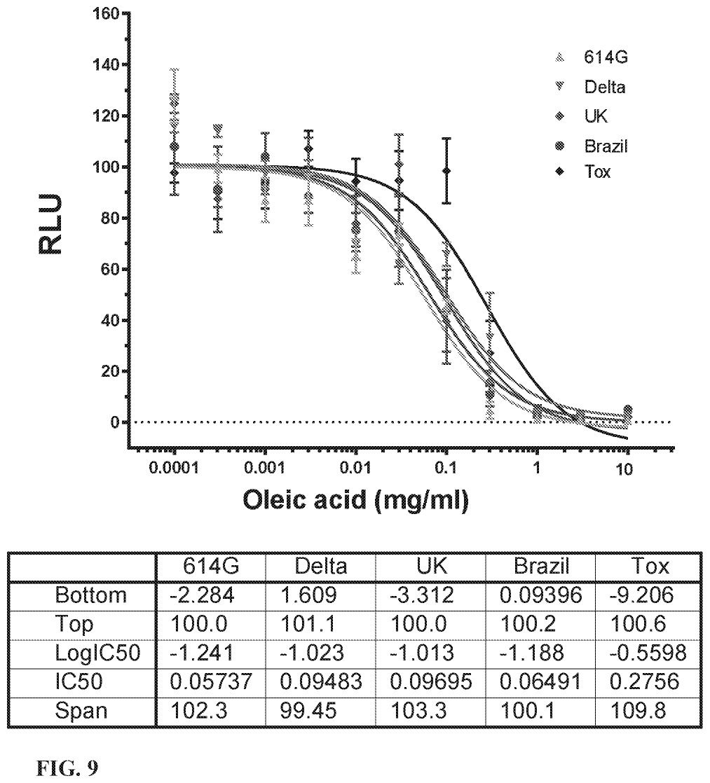

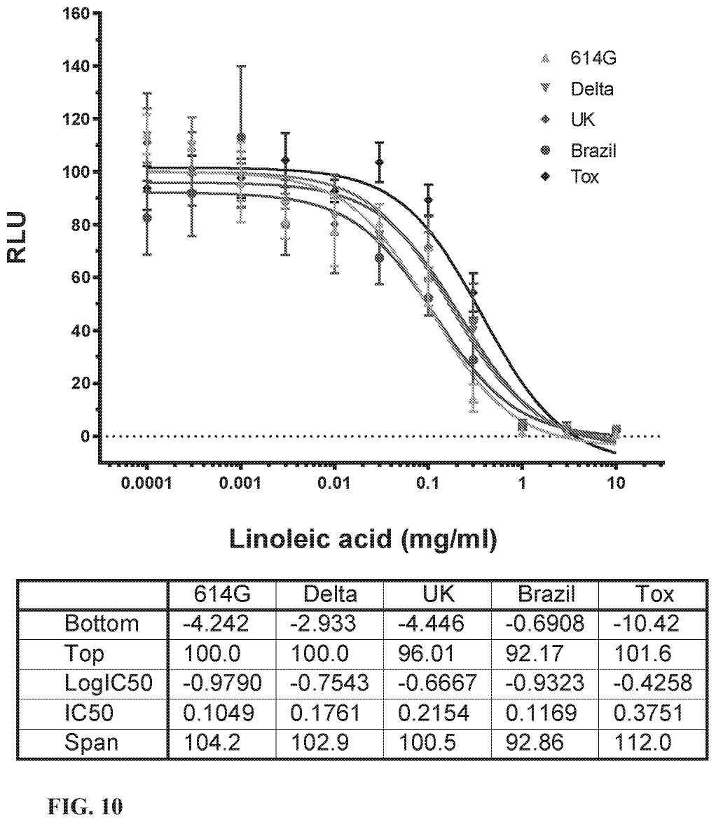

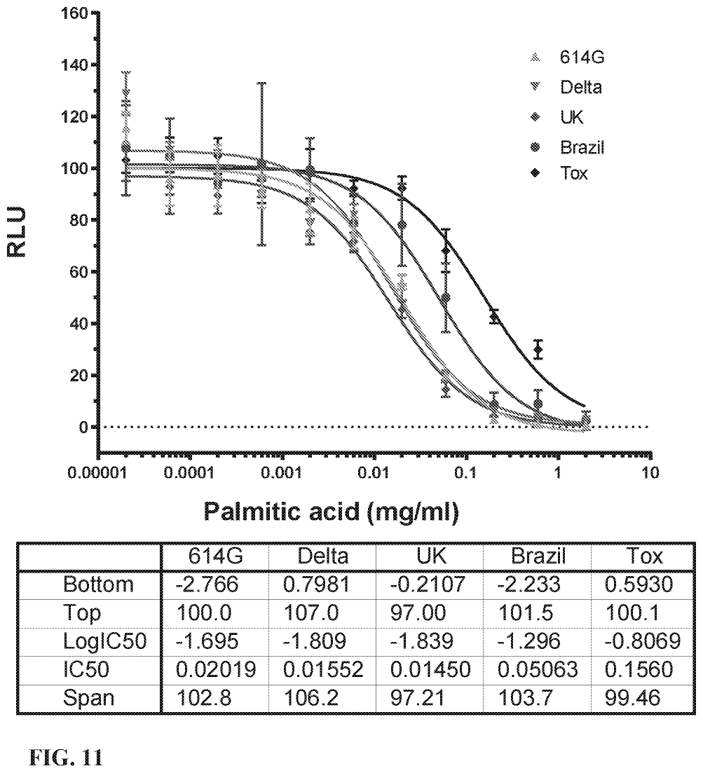

The data was normalized as the percentage of the highest reading (low concentration of each compound or no compound) of each compound (relative luciferase activity, RLU). They were used to draw the dose response curves against the compound concentrations. IC50 values were calculated based on curve fitting in GraphPad Prism. In each figure, one compound was tested against 4 SARS-CoV2-PPs. The results are shown in FIGS. 7-11, which demonstrate the effect of blackseed oil, thymoquinone and fatty acids to blocks SARS-CoV-2-PP infecting the ACE2 expression cells, in which the x-axis is the compound concentration and the y-axis is the relative luminescence unit (RLU), reflecting the luciferase activity and the viral infectivity.

The following combinations of the compounds with the 4 different SARS-CoV2-PPs were then tested:
6. Thymoquinone+Oleic acid
7. Thymoquinone+Linoleic Acid
8. Thymoquinone+Palmitic acid
9. Oleic acid+Linoleic acid
10. Oleic acid+Palmitic acid
11. Linoleic acid+Palmitic acid

Based on the results in FIG. 7, it was decided to perform the dose response assays of Oleic acid, Linoleic Acid and Palmitic acid in the presence of 0.05 μM Thymoquinone; dose response assays of Linoleic Acid and Palmitic acid in the presence of 0.01 mg/ml of Oleic acid; dose response assays of Palmitic acid in the presence of 0.01 mg/ml Linoleic Acid.

The day before the infection, 7.5K HEK293-ACE2 cells were plated into a 384-well white clear plate (Cat #353963, Corning Life Sciences) precoated with Poly D Lysine (Cat #3439-100-1, Trevigen, Inc) in 15 μl culture medium (DMEM+10% FetalClone II Serum, Cat #SH3006603, Fisher Scientific). The cell plate was placed in a CO2 incubator (37° C.).

In a manner analogous to that disclosed above, on the second day, the compounds to be tested were diluted in the culture medium on a 96-well compound plate, as shown below in tables 7-12. They are 5× of the final concentrations.

TABLE 7
An example of Oleic Acid concentrations in a compound
dilution plate in the presence of 0.25 μM Thymoquinone

| Sample # | 1 | 2 | 3 | 4 | 5 | 6 | 7 | 8 | 9 | 10 | 11 | 12 |
|---|---|---|---|---|---|---|---|---|---|---|---|---|
| Thymoquinone | 0.25 | | | | | | | | | | | |
| (μM) | | | | | | | | | | | | |
| Oleic acid | 50 | 15 | 5 | 1.5 | 0.5 | 0.15 | .05 | .015 | .005 | .0015 | .0005 | 0 |
| (mg/ml) | | | | | | | | | | | | |

TABLE 8
An example of Linoleic Acid concentrations in a compound
dilution plate in the presence of 0.25 μM Thymoquinone

| Sample # | 1 | 2 | 3 | 4 | 5 | 6 | 7 | 8 | 9 | 10 | 11 | 12 |
|---|---|---|---|---|---|---|---|---|---|---|---|---|
| Thymoquinone | 0.25 | | | | | | | | | | | |
| (μM) | | | | | | | | | | | | |
| Linoleic acid | 50 | 15 | 5 | 1.5 | 0.5 | .15 | .05 | .015 | .005 | .0015 | .0005 | 0 |
| (mg/ml) | | | | | | | | | | | | |

TABLE 9
An example of Palmitic Acid concentrations in a compound
dilution plate in the presence of 0.25 μM Thymoquinone

| Sample # | 1 | 2 | 3 | 4 | 5 | 6 | 7 | 8 | 9 | 10 | 11 | 12 |
|---|---|---|---|---|---|---|---|---|---|---|---|---|
| Thymoquinone | 0.25 | | | | | | | | | | | |
| (μM) | | | | | | | | | | | | |
| Palmitic acid | 10 | 3 | 1 | 0.3 | 0.1 | .03 | .01 | .003 | .001 | .0003 | .0001 | 0 |
| (mg/ml) | | | | | | | | | | | | |

TABLE 10
An example of Linoleic Acid concentrations in a compound
dilution plate in the presence of 0.05 mg/ml Oleic acid

| Sample # | 1 | 2 | 3 | 4 | 5 | 6 | 7 | 8 | 9 | 10 | 11 | 12 |
|---|---|---|---|---|---|---|---|---|---|---|---|---|
| Oleic acid | 0.05 | | | | | | | | | | | |
| (mg/ml) | | | | | | | | | | | | |
| Linoleic acid | 50 | 15 | 5 | 1.5 | 0.5 | .15 | .05 | .015 | .005 | .0015 | .0005 | 0 |
| (mg/ml) | | | | | | | | | | | | |

TABLE 11
An example of Palmitic Acid concentrations in a compound
dilution plate in the presence of 0.05 mg/ml Oleic acid

| Sample # | 1 | 2 | 3 | 4 | 5 | 6 | 7 | 8 | 9 | 10 | 11 | 12 |
|---|---|---|---|---|---|---|---|---|---|---|---|---|
| Oleic acid (mg/ml) | 0.05 | | | | | | | | | | | |
| Palmitic acid (mg/ml) | 10 | 3 | 1 | 0.3 | 0.1 | .03 | .01 | .003 | .001 | .0003 | .0001 | 0 |

TABLE 12
An example of Palmitic Acid concentrations in a compound dilution
plate in the presence of 0.05 mg/ml Linoleic acid

| Sample # | 1 | 2 | 3 | 4 | 5 | 6 | 7 | 8 | 9 | 10 | 11 | 12 |
|---|---|---|---|---|---|---|---|---|---|---|---|---|
| Linoleic acid (mg/ml) | 0.05 | | | | | | | | | | | |
| Palmitic acid (mg/ml) | 10 | 3 | 1 | 0.3 | 0.1 | .03 | .01 | .003 | .001 | .0003 | .0001 | 0 |

Eighty (80) td of SARS-CoV-2 MLV pseudoviruse particles (pp) were mixed with 32 μl of each testing compound prepared above and incubated at 37° C. for 10 min. After the medium in each well of 384-well cell plate was removed, 17.5 μl of each compound-pp mixture was added into each well.

TABLE 13
An example of final concentrations of Oleic acid, Linoleic acid and Palmitic
acid in the presence of the other compound on the 384-well assay plate

| | 1 | 2 | 3 | 4 | 5 | 6 | 7 | 8 | 9 | 10 | |
|---|---|---|---|---|---|---|---|---|---|---|---|
| A | 10 | 10 | 3 | 3 | 1 | 1 | .3 | .3 | .1 | .1 | Oleic acid (mg/ml + |
| | | | | | | | | | | | 0.05 μM |
| | | | | | | | | | | | Thymoquinone |
| B | 10 | 10 | 3 | 3 | 1 | 1 | .3 | .3 | .1 | .1 | Oleic acid (mg/ml + |
| | | | | | | | | | | | 0.05 μM |
| | | | | | | | | | | | Thymoquinone |
| C | 10 | 10 | 3 | 3 | 1 | 1 | .3 | .3 | .1 | .1 | Linoleic acid (mg/ml + |
| | | | | | | | | | | | 0.05 μM |
| | | | | | | | | | | | Thymoquinone |
| D | 10 | 10 | 3 | 3 | 1 | 1 | .3 | .3 | .1 | .1 | Linoleic acid (mg/ml + |
| | | | | | | | | | | | 0.05 μM |
| | | | | | | | | | | | Thymoquinone |
| E | 2 | 2 | .6 | .6 | .2 | .2 | .06 | .06 | .02 | .02 | Palmitic acid (mg/ml + |
| | | | | | | | | | | | 0.05 μM |
| | | | | | | | | | | | Thymoquinone |
| F | 2 | 2 | .6 | .6 | .2 | .2 | .06 | .06 | .02 | .02 | Palmitic acid (mg/ml + |
| | | | | | | | | | | | 0.05 μM |
| | | | | | | | | | | | Thymoquinone |
| G | 10 | 10 | 3 | 3 | 1 | 1 | .3 | .3 | .1 | .1 | Linoleic Acid (mg/ml) + |
| | | | | | | | | | | | 0.01 mg/ml Oleic |
| | | | | | | | | | | | acid |
| H | 10 | 10 | 3 | 3 | 1 | 1 | .3 | .3 | .1 | .1 | Linoleic Acid (mg/ml) + |
| | | | | | | | | | | | 0.01 mg/ml Oleic |
| | | | | | | | | | | | acid |
| I | 2 | 2 | .6 | .6 | .2 | .2 | .06 | .06 | .02 | .02 | Palmitic Acid (mg/ml) + |
| | | | | | | | | | | | 0.01 mg/ml Oleic |
| | | | | | | | | | | | acid |
| J | 2 | 2 | .6 | .6 | .2 | .2 | .06 | .06 | .02 | .02 | Palmitic Acid (mg/ml) + |
| | | | | | | | | | | | 0.01 mg/ml Oleic |
| | | | | | | | | | | | acid |
| K | 2 | 2 | .6 | .6 | .2 | .2 | .06 | .06 | .02 | .02 | Palmitic Acid (mg/ml) + |
| | | | | | | | | | | | 0.01 mg/ml Linoleic |
| | | | | | | | | | | | acid |
| L | 2 | 2 | .6 | .6 | .2 | .2 | .06 | .06 | .02 | .02 | Palmitic Acid (mg/ml) + |
| | | | | | | | | | | | 0.01 mg/ml Linoleic |
| | | | | | | | | | | | acid |

| | 11 | 12 | 13 | 14 | 15 | 16 | 17 | 18 | 19 | 20 | 21 | 22 | 23 | 24 |
|---|---|---|---|---|---|---|---|---|---|---|---|---|---|---|
| A | .03 | .03 | .01 | .01 | .003 | .003 | .001 | .001 | 3E−04 | 3E−04 | 1E−04 | 1E−04 | 0 | 0 |
| B | .03 | .03 | .01 | .01 | .003 | .003 | .001 | .001 | 3E−04 | 3E−04 | 1E−04 | 1E−04 | 0 | 0 |
| C | .03 | .03 | .01 | .01 | .003 | .003 | .001 | .001 | 3E−04 | 3E−04 | 1E−04 | 1E−04 | 0 | 0 |
| D | .03 | .03 | .01 | .01 | .003 | .003 | .001 | .001 | 3E−04 | 3E−04 | 1E−04 | 1E−04 | .0 | 0 |
| E | .006 | .006 | .002 | .002 | 6E−04 | 6E−04 | 2E−04 | 2E−04 | 6E−05 | 6E−05 | 2E−05 | 2E−05 | 0 | 0 |
| F | .006 | .006 | .002 | .002 | 6E−04 | 6E−04 | 2E−04 | 2E−04 | 6E−05 | 6E−05 | 2E−05 | 2E−05 | 0 | 0 |
| G | .03 | .03 | .01 | .01 | .003 | .003 | .001 | .001 | 3E−04 | 3E−04 | 1E−04 | 1E−04 | 0 | 0 |
| H | .03 | .03 | .01 | .01 | .003 | .003 | .001 | .001 | 3E−04 | 3E−04 | 1E−04 | 1E−04 | 0 | 0 |
| I | .006 | .006 | .002 | .002 | 6E−04 | 6E−04 | 2E−04 | 2E−04 | 6E−05 | 6E−05 | 2E−05 | 2E−05 | 0 | 0 |
| J | .006 | .006 | .002 | .002 | 6E−04 | 6E−04 | 2E−04 | 2E−04 | 6E−05 | 6E−05 | 2E−05 | 2E−05 | 0 | 0 |
| K | .006 | .006 | .002 | .002 | 6E−04 | 6E−04 | 2E−04 | 2E−04 | 6E−05 | 6E−05 | 2E−05 | 2E−05 | 0 | 0 |
| L | .006 | .006 | .002 | .002 | 6E−04 | 6E−04 | 2E−04 | 2E−04 | 6E−05 | 6E−05 | 2E−05 | 2E−05 | 0 | 0 |

The plate was centrifuged at 54 g for 15 min at 4° C. and additional 7.5 id of the culture medium was then added into each well. The total final volume in each well was 25 id. The cells were then incubated at 37° C. for 42 hr. Luciferase activities were measured with Firefly Luciferase Assay Kit (CB-80552-010, Codex BioSolutions Inc). IC50 values were calculated based on curve fitting in GraphPad Prism.

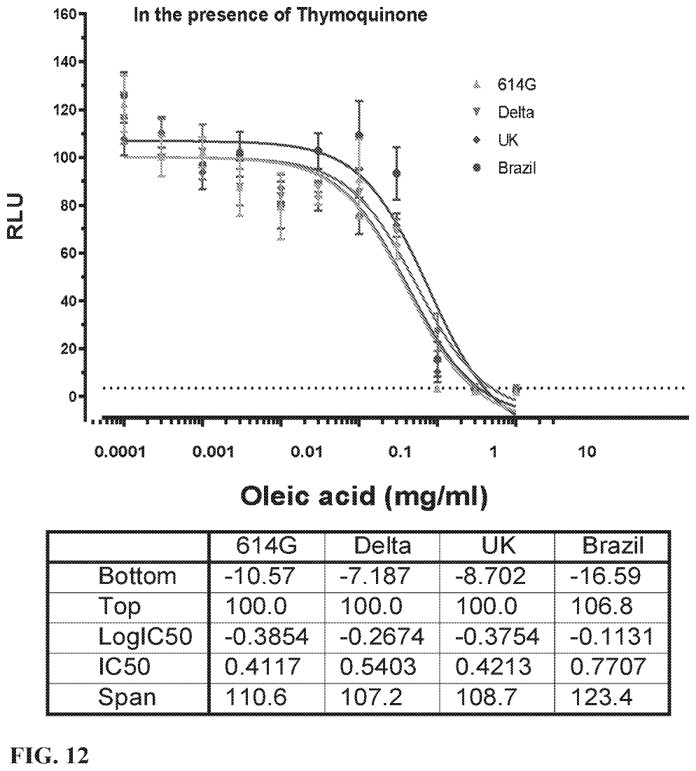

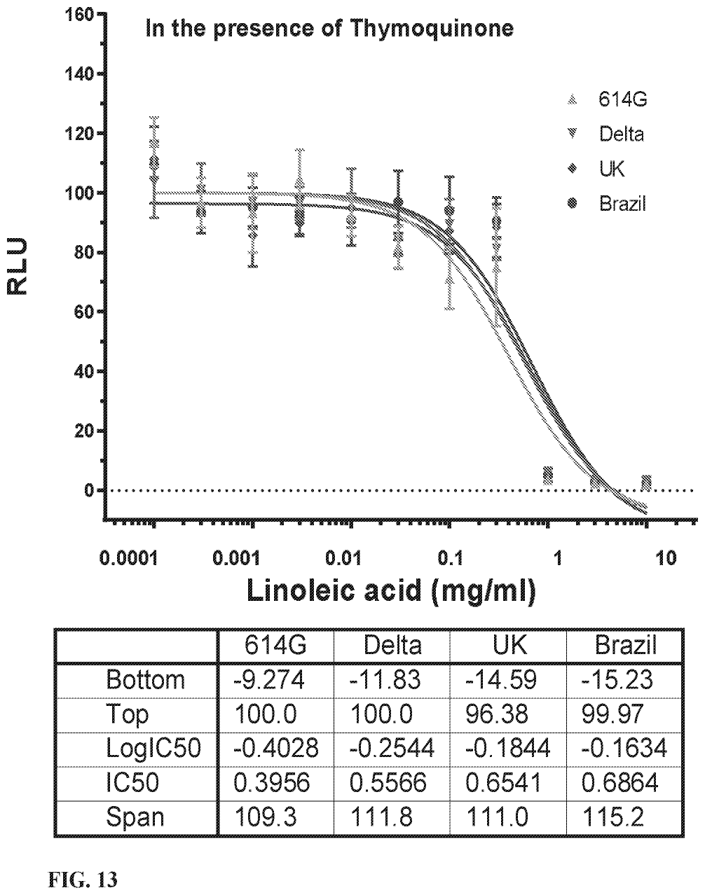

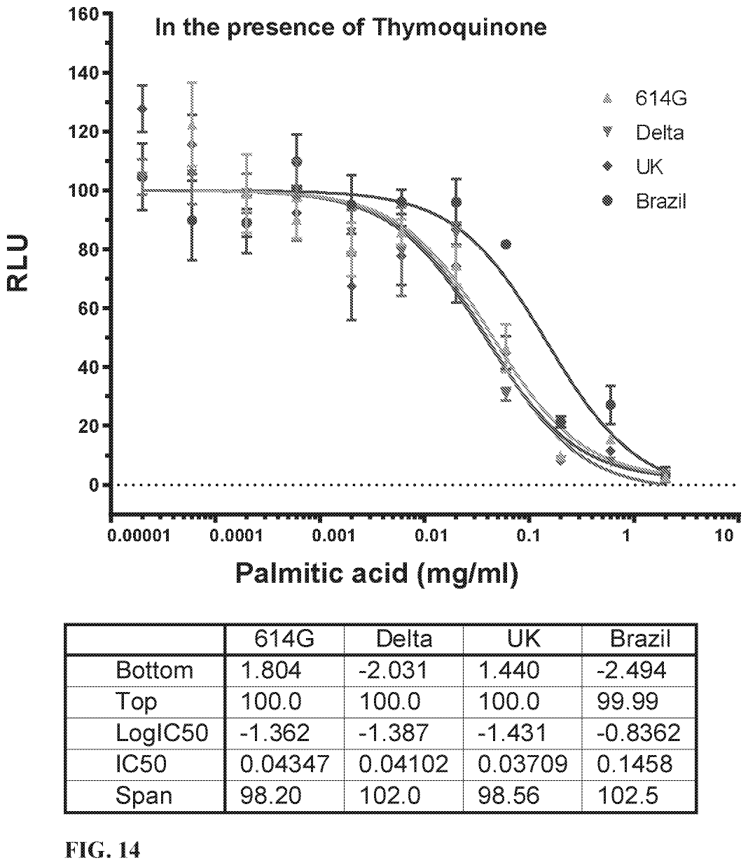

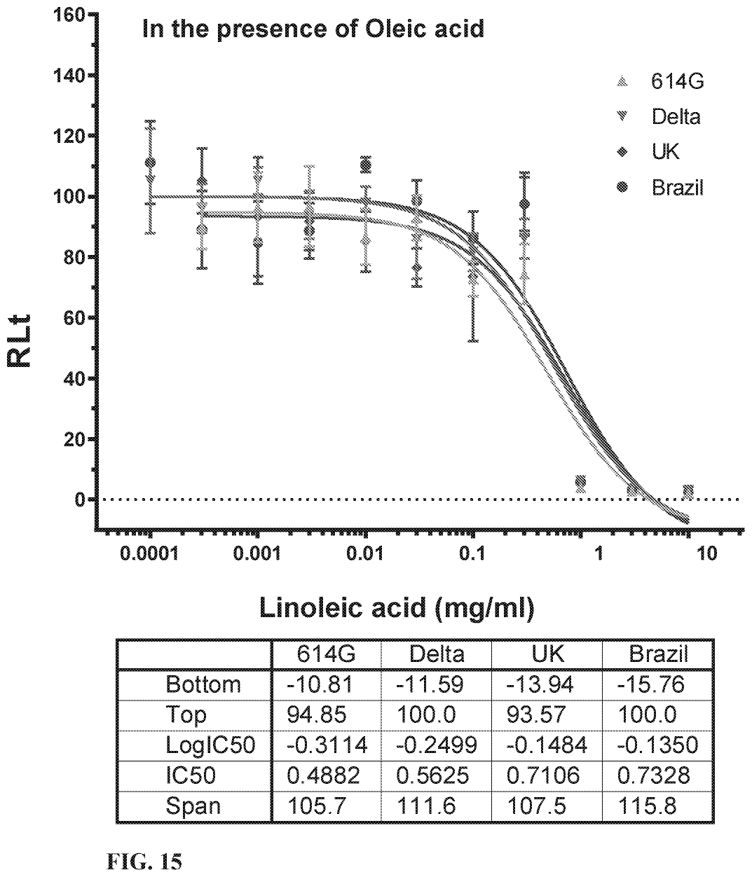

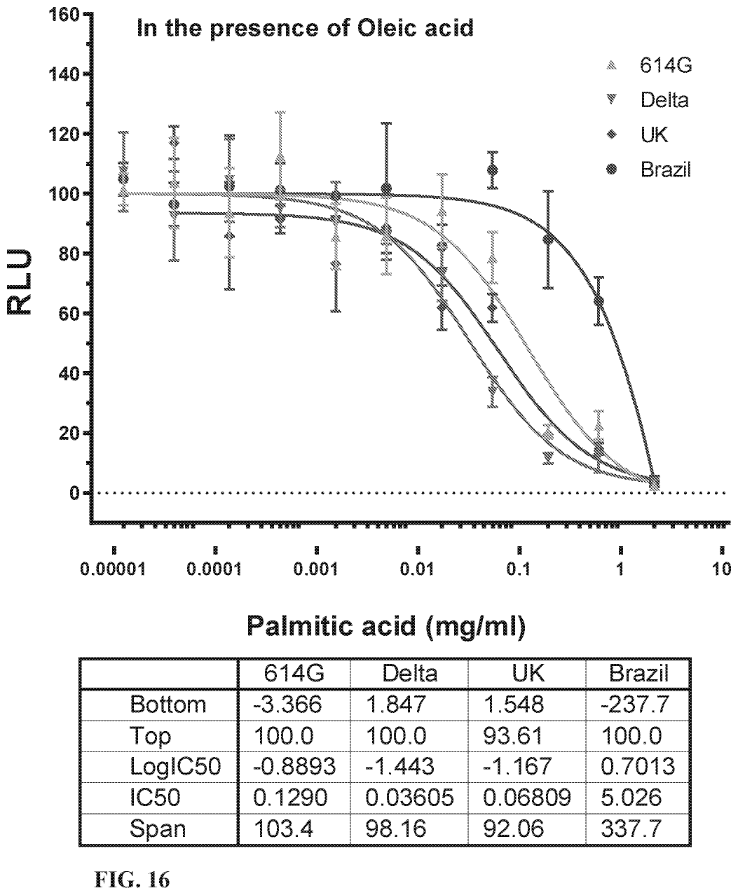

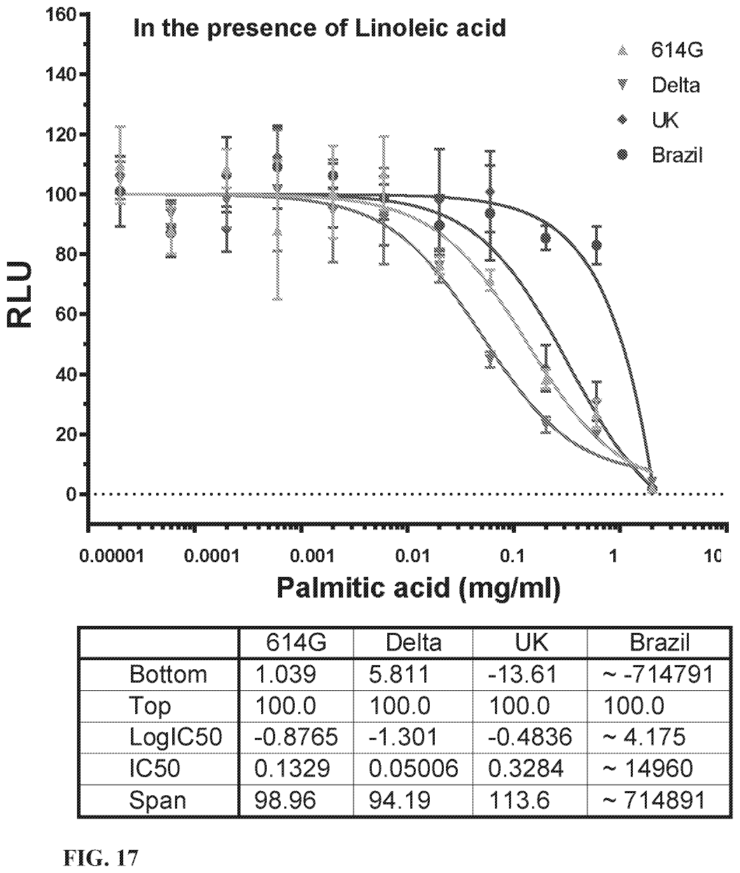

The data was normalized as the percentage of the highest reading (low concentration of each compound or no compound) of each compound (relative luciferase activity, RLU). They were used to draw the dose response curves against the compound concentrations. IC50 values were calculated based on curve fitting in GraphPad Prism. In each figure, each compound combination was tested against 4 SARS-CoV2-PPs. The results are set forth in FIGS. 12-17, which depict the effect of oleic acid, linoleic acid, palmitic acid in combination with Thymoquinone or with another fatty acid to block SARS-CoV-2-PP infecting the ACE2 expression cells. X-axis depicts the compound concentration and the Y-axis depicts the relative luminescence unit (RLU), reflecting the luciferase activity and the viral infectivity.

In addition to thymoquinone, palmitic acid is a good inhibitor for SARS-CoV-2-PP infection. It showed a difference between the Brazil variant and the other variants. When combined with Oleic acid or Linoleic acid, it showed a different effect on Delta, 614G, UK and Brazil variants, with the strongest inhibition on the Delta variant.

### Example 3: In Vitro Viral Studies—Black Seed Oil and/or Thymoquinone

By a similar procedure as described in Example 2, samples of black seed oil only of varying concentrations, thymoquinone only of varying molarity, black seed oil of varying concentrations with 2 μM of thymoquinone and thymoquinone of varying molarity with 2% black seed oil were respectively prepared in a compound dilution plates. Examples of prepared samples are shown below in Tables 1-4:

TABLE 14
An example of Black seed Oil concentrations in a compound dilution plate.

| Sample # | 1 | 2 | 3 | 4 | 5 | 6 | 7 | 8 | 9 | 10 | 11 | 12 |
|---|---|---|---|---|---|---|---|---|---|---|---|---|
| Black seed Oil | 100% | 30% | 10% | 3% | 1% | 0.03% | 0.01% | 0.03% | 0.01% | 0.003% | 0.0001% | 0 |

TABLE 15
An example of Thymoquinone concentrations in a compound dilution plate.

| Sample # | 1 | 2 | 3 | 4 | 5 | 6 | 7 | 8 | 9 | 10 | 11 | 12 |
|---|---|---|---|---|---|---|---|---|---|---|---|---|
| Thymoquinone (μM) | 500 | 150 | 50 | 15 | 5 | 1.5 | 0.5 | 0.15 | 0.05 | 0.015 | 0.005 | 0 |

TABLE 16
An example of Black seed Oil concentrations in a compound
dilution plate in the presence of 2 μM Thymoquinone.

| Sample # | 1 | 2 | 3 | 4 | 5 | 6 | 7 | 8 | 9 | 10 | 11 | 12 |
|---|---|---|---|---|---|---|---|---|---|---|---|---|
| Thymoquinone (μM) | 2 | | | | | | | | | | | |
| Black seed Oil | 100% | 30% | 10% | 3% | 1% | 0.3% | 0.1% | 0.03% | 0.01% | 0.003% | 0.0001% | 0 |

TABLE 17
An example of Thymoquinone concentrations in a compound
dilution plate in the presence of 2% Black seed Oil.

| Sample # | 1 | 2 | 3 | 4 | 5 | 6 | 7 | 8 | 9 | 10 | 11 | 12 |
|---|---|---|---|---|---|---|---|---|---|---|---|---|
| Black seed Oil | 2% | | | | | | | | | | | |
| Thymoquinone (μM) | 500 | 150 | 50 | 15 | 5 | 1.5 | 0.5 | 0.15 | 0.05 | 0.015 | 0.005 | 0 |

Eighty (80) id of SARS-CoV-2 MLV psuedoparticles where mixed with 32 μl of each sample, prepared as described above, and incubated at 37° C. for 10 min. After the medium in each well of 384-well cell plate was removed, 17.5 μl of each compound-pp mixture was added into each well. Examples of final concentrations of black seed oil and thymoquinone on the 384-well assay plate are set forth below in Table 5:

TABLE 18
An example of final concentrations of Black seed
Oil and Thymoquinone on the 384-well assay plate.

| | 1 | 2 | 3 | 4 | 5 | 6 | 7 | 8 | 9 | 10 | 11 | 12 | 13 | |
|---|---|---|---|---|---|---|---|---|---|---|---|---|---|---|
| A | 20% | 20% | 6% | 6% | 2% | 2% | 1% | 1% | 0% | 0% | 0.1% | 0.1% | 0.0% | Black seed Oil |
| B | 20% | 20% | 6% | 6% | 2% | 2% | 1% | 1% | 0% | 0% | 0.1% | 0.1% | 0.0% | Black seed Oil |
| C | 100 | 100 | 30 | 30 | 10 | 10 | 3 | 3 | 1 | 1 | 0.3 | 0.3 | 0.1 | Thymoquinone |
| | | | | | | | | | | | | | | (μM) |
| D | 100 | 100 | 30 | 30 | 10 | 10 | 3 | 3 | 1 | 1 | 0.3 | 0.3 | 0.1 | Thymoquinone |
| | | | | | | | | | | | | | | (μM) |
| E | 20% | 20% | 6% | 6% | 2% | 2% | 1% | 1% | 0% | 0% | 0.1% | 0.1% | 0.0% | Black seed Oil |
| | | | | | | | | | | | | | | with 0.4 μM |
| | | | | | | | | | | | | | | Thymoquinone |
| F | 20% | 20% | 6% | 6% | 2% | 2% | 1% | 1% | 0% | 0% | 0.1% | 0.1% | 0.0% | Black seed Oil |
| | | | | | | | | | | | | | | with 0.4 μM |
| | | | | | | | | | | | | | | Thymoquinone |
| G | 100 | 100 | 30 | 30 | 10 | 10 | 3 | 3 | 1 | 1 | 0.3 | 0.3 | 0.1 | Thymoquinone |
| | | | | | | | | | | | | | | (μM) with 0.4% |
| | | | | | | | | | | | | | | Black seed Oil |
| H | 100 | 100 | 30 | 30 | 10 | 10 | 3 | 3 | 1 | 1 | 0.3 | 0.3 | 0.1 | Thymoquinone |
| | | | | | | | | | | | | | | (μM) with 0.4% |
| | | | | | | | | | | | | | | Black seed Oil |

| | 14 | 15 | 16 | 17 | 18 | 19 | 20 | 21 | 22 | 23 | 24 |
|---|---|---|---|---|---|---|---|---|---|---|---|
| A | 0.0% | 0.01% | 0.01% | 0.00% | 0.00% | 0.001% | 0.001% | 0.0002% | 0.0002% | 0 | 0 |
| B | 0.0% | 0.01% | 0.01% | 0.00% | 0.00% | 0.001% | 0.001% | 0.0002% | 0.0002% | 0 | 0 |
| C | 0.1 | 0.03 | 0.03 | 0.01 | 0.01 | 0.003 | 0.003 | 0.001 | 0.001 | 0 | 0 |
| D | 0.1 | 0.03 | 0.03 | 0.01 | 0.01 | 0.003 | 0.003 | 0.001 | 0.001 | 0 | 0 |
| E | 0.0% | 0.01% | 0.01% | 0.00% | 0.00% | 0.001% | 0.001% | 0.0002% | 0.0002% | 0 | 0 |
| F | 0.0% | 0.01% | 0.01% | 0.00% | 0.00% | 0.001% | 0.001% | 0.0002% | 0.0002% | 0 | 0 |
| G | 0.1 | 0.03 | 0.03 | 0.01 | 0.01 | 0.003 | 0.003 | 0.001 | 0.001 | 0 | 0 |
| H | 0.1 | 0.03 | 0.03 | 0.01 | 0.01 | 0.003 | 0.003 | 0.001 | 0.001 | 0 | 0 |

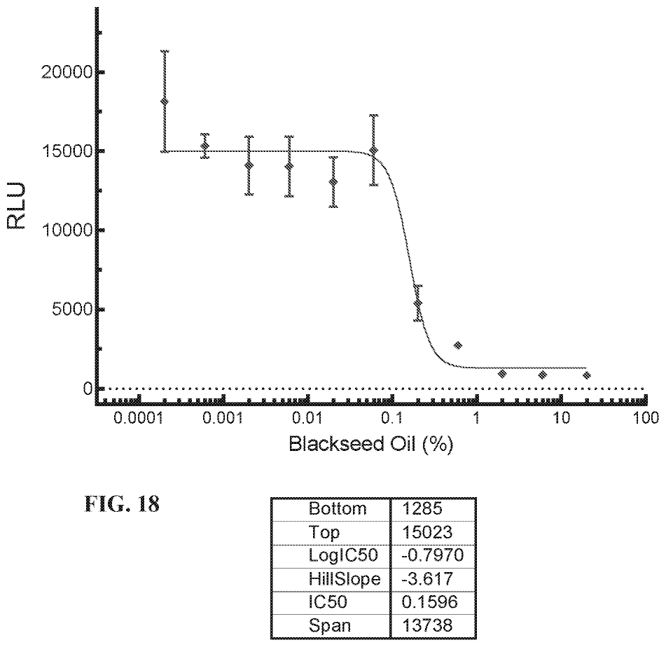

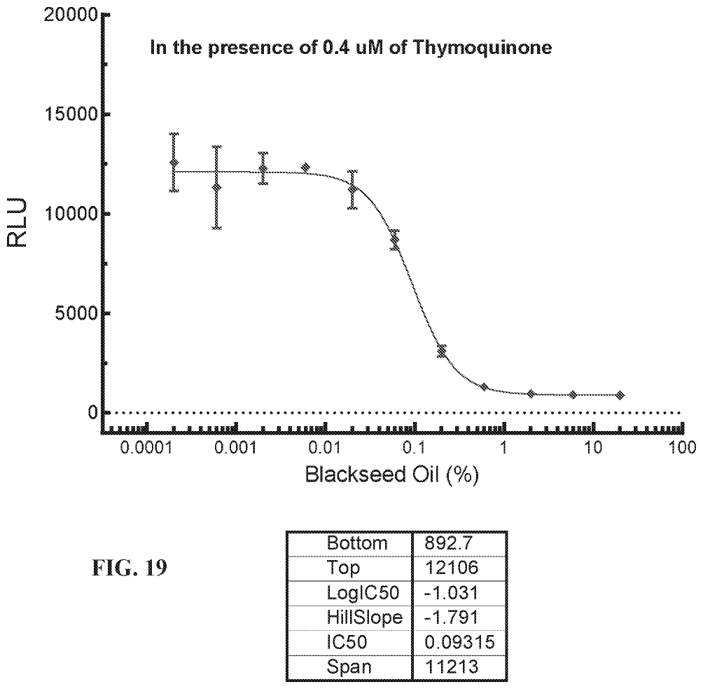

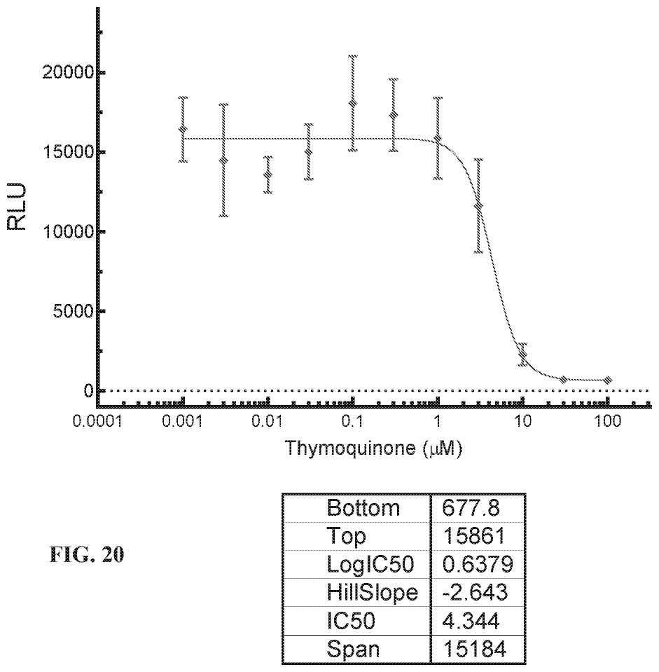

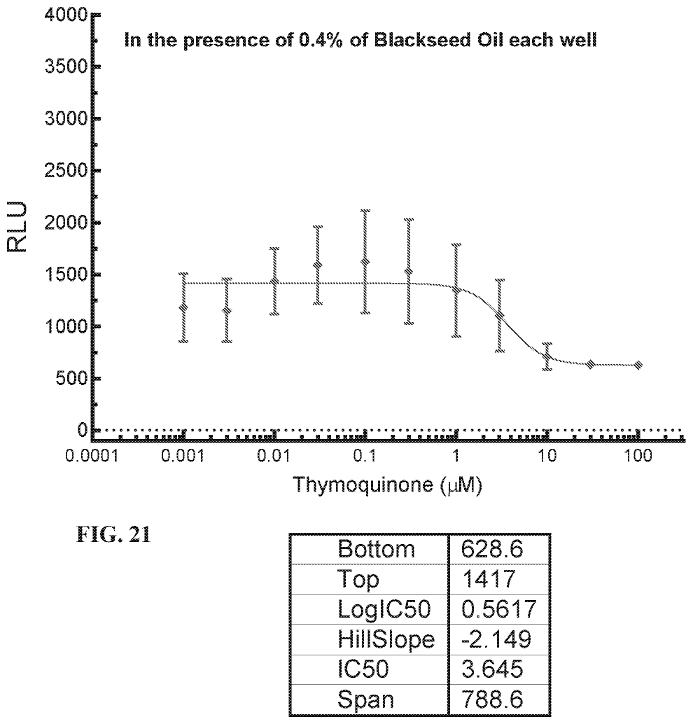

The plate was centrifuged at 54 g for 15 min at 4° C. and additional 7.5 μl of the culture medium was then added into each well. The total final volume in each well was 25 μl. The cells were then incubated at 37° C. for 42 hr. Luciferase activities were measured with Firefly Luciferase Assay Kit (CB-80552-010, Codex BioSolutions Inc). IC50 values were calculated based on curve fitting in GraphPad Prism. Results were obtained as shown in FIGS. 18-21, which demonstrates the effect of black seed oil and thymoquinone to block SARS-CoV-2-PP from infecting the cells, in which the X-axis depicts the sample concentration and the y-axis depicts the relative luminescence unit (RLU), reflecting the luciferase activity and the viral infectivity.

The results indicate that black seed oil and thymoquinone block the viral infection. However, it was found that at high concentrations, black seed oil and thymoquinone can cause cell death, which indicates that both may have cell toxicity. Nevertheless, toxic doses of black seed oil are many fold higher than therapeutic doses.

To confirm these results, a cell grow assay was performed in the presence of black seed oil or Thymoquinone with Codex's EnerCount cell growth assay kit which measures the ATP levels inside the cells (Cat #CB-80551-010, Codex BioSolutions). The day before the infection, 7.5K Expi-293F-ACE2 cells were plated into each well of a 384-well white clear plate (Cat #353963, Corning Life Sciences) precoated with Poly D Lysine (Cat #3439-100-1, Trevigen, Inc) in 15 μl culture medium (DMEM+10% FetalClone II Serum, Cat #SH3006603, Fisher Scientific). The cell plate was placed in a CO₂ incubator (37° C.).

On the 2nd day, the compounds to be tested were diluted in the culture medium on a 96-well compound plate as shown above in tables 14-17. They are 5× of the final concentrations.

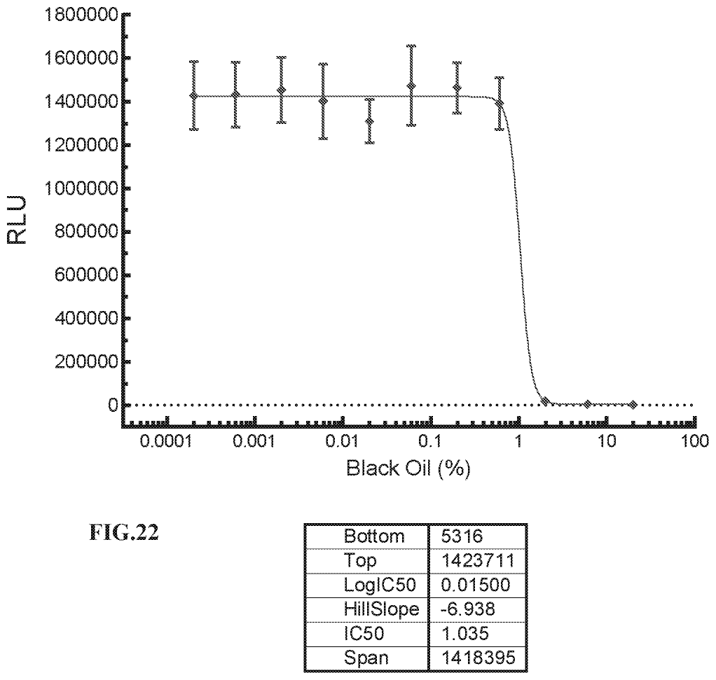

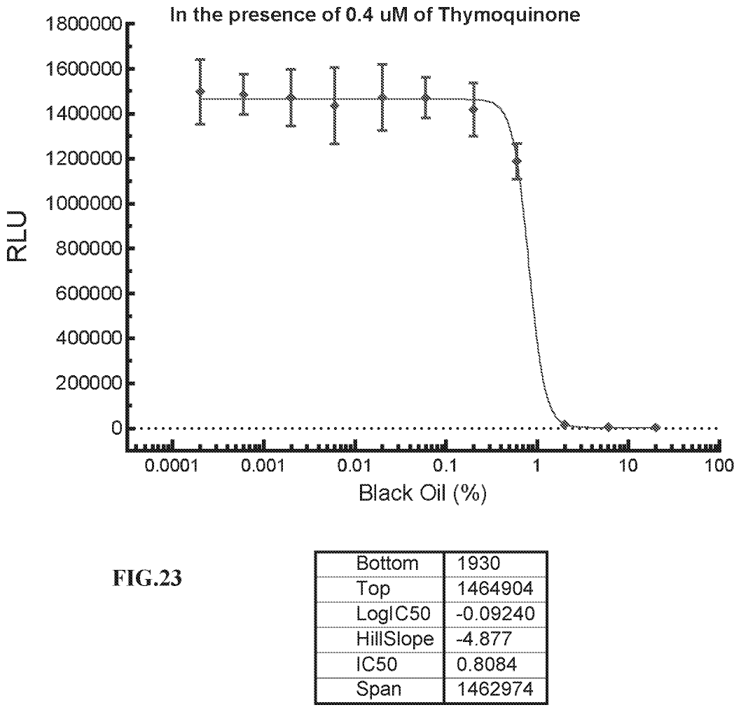

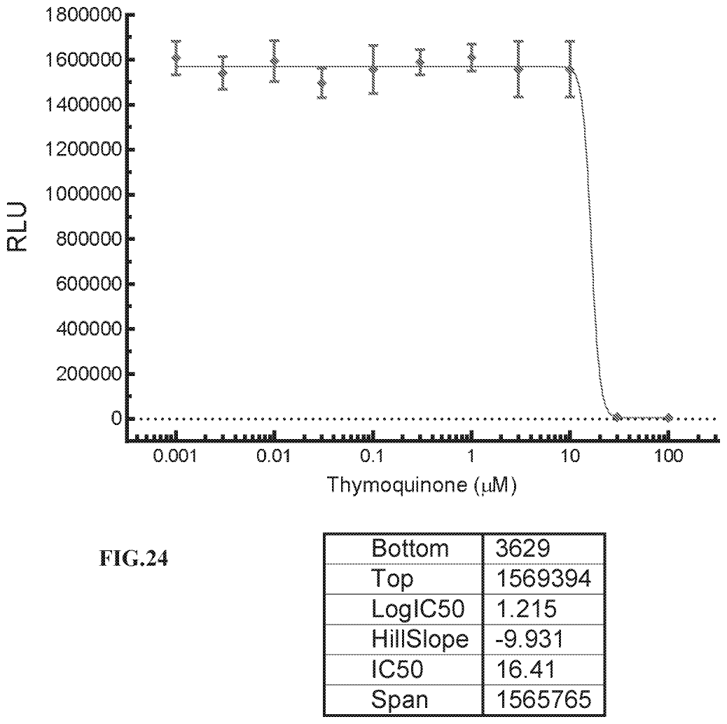

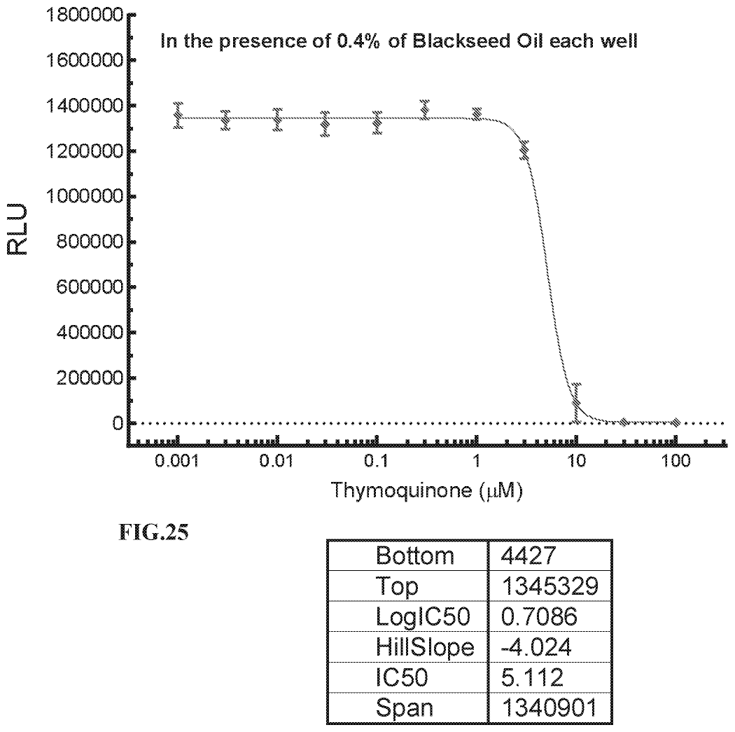

The plate was taken out of the incubator and the medium in each well was removed. Twenty (20) μl culture medium (DMEM+10% FetalClone II Serum, Cat #SH3006603, Fisher Scientific) was added back into each well. 5 μl of the compound prepared as shown in Table 1-4 was added into each well. The plate was centrifuged at 54 g for 15 min at 4° C. The cells were then incubated at 37° C. for 42 hr. Luciferase activities were measured with Codex's EnerCount cell growth assay kit. The results are shown in FIGS. 22-25. With reference to FIGS. 18-25, one can conclude that Black seed oil and Thymoquinone can block the SARS-CoV-2-MLV-PP infection without any cell toxicity (e.g., black seed oil (0.06%-0.6%) and thymoquinone (1-10 μM). A synergistic effect between black seed oil and thymoquinone is believed to exist. Adding thymoquinone to black seed oil reduced the IC₅₀ from 0.16 to 0.09 suggesting about 40.0% increase in effectiveness. Similarly, adding black seed oil to the thymoquinone reduced the IC₅₀ from 4.3 to 3.6 suggesting an increased effectiveness of about 16.0%.

### Example 4: In Vitro Viral Studies—Addition of Temsavir and Dolutegravir

By a process as generally described in Example 2, Temsavir and Dolutegravir was tested with or without Black seed oil or Thymoquinone. The day before the infection, 7.5K Expi-293F-ACE2 cells were plated into each well of a 384-well white clear plate (Cat #353963, Corning Life Sciences) precoated with Poly D Lysine (Cat #3439-100-1, Trevigen, Inc) in 15 μl culture medium (DMEM+10% FetalClone II Serum, Cat #SH3006603, Fisher Scientific). The cell plate was placed in a CO2 incubator (37° C.). On the 2nd day, the compounds to be tested were diluted in the culture medium on a 96-well compound plate as shown in tables 6-11. They are 5× of the final concentrations.

Examples of prepared samples are shown below in Tables 19-24:

TABLE 19
An example of Temsavir concentrations in a compound dilution plate.

| Sample # | 1 | 2 | 3 | 4 | 5 | 6 | 7 | 8 | 9 | 10 | 11 | 12 |
|---|---|---|---|---|---|---|---|---|---|---|---|---|
| Temsavir (μM) | 500 | 150 | 50 | 15 | 5 | 1.5 | 0.5 | 0.15 | 0.05 | 0.015 | 0.005 | 0 |

TABLE 20
An example of Dolutegravir concentrations in a compound dilution plate.

| Sample # | 1 | 2 | 3 | 4 | 5 | 6 | 7 | 8 | 9 | 10 | 11 | 12 |
|---|---|---|---|---|---|---|---|---|---|---|---|---|
| Dolutegravir (μM) | 50 | 15 | 5 | 1.5 | 0.5 | 0.15 | 0.05 | 0.015 | 0.005 | 0.0015 | 0.0005 | 0 |

TABLE 21
An example of Temsavir concentrations in a compound dilution
plate in the presence of 0.4% Black seed Oil.

| Sample # | 1 | 2 | 3 | 4 | 5 | 6 | 7 | 8 | 9 | 10 | 11 | 12 |
|---|---|---|---|---|---|---|---|---|---|---|---|---|
| Black seed Oil | 0.4% | | | | | | | | | | | |
| Temsavir (μM) | 500 | 150 | 50 | 15 | 5 | 1.5 | 0.5 | 0.15 | 0.05 | 0.015 | 0.005 | 0 |

TABLE 22
An example of Dolutegravir concentrations in a compound
dilution plate in the presence of 0.4% Black seed Oil.

| Sample # | 1 | 2 | 3 | 4 | 5 | 6 | 7 | 8 | 9 | 10 | 11 | 12 |
|---|---|---|---|---|---|---|---|---|---|---|---|---|
| Black seed Oil | 0.4% | | | | | | | | | | | |
| Dolutegravir (μM) | 50 | 15 | 5 | 1.5 | 0.5 | 0.15 | 0.05 | 0.015 | 0.0005 | 0.0015 | 0.0005 | 0 |

TABLE 23
An example of Temsavir concentrations in a compound
dilution plate in the presence of 5 μM Thymoquinone.

| Sample # | 1 | 2 | 3 | 4 | 5 | 6 | 7 | 8 | 9 | 10 | 11 | 12 |
|---|---|---|---|---|---|---|---|---|---|---|---|---|
| Thymoquinone (μM) | 5 | | | | | | | | | | | |
| Temsavir (μM) | 500 | 150 | 50 | 15 | 5 | 1.5 | 0.5 | 0.15 | 0.05 | 0.015 | 0.005 | 0 |

TABLE 24
An example of Dolutegravir concentrations in a compound
dilution plate in the presence of 5 (μM) Thymoquinone.

| Sample # | 1 | 2 | 3 | 4 | 5 | 6 | 7 | 8 | 9 | 10 | 11 | 12 |
|---|---|---|---|---|---|---|---|---|---|---|---|---|
| Thymoquinone (μM) | 5 | | | | | | | | | | | |
| Dolutegravir (μM) | 50 | 15 | 5 | 1.5 | 0.5 | 0.15 | 0.05 | 0.015 | 0.005 | 0.0015 | 0.0005 | 0 |

Eighty (80) μ1 of SARS-CoV-2 MLV psuedoparticles where mixed with 32 μl of each sample, prepared as described above, and incubated at 37° C. for 10 min. After the medium in each well of 384-well cell plate was removed, 17.5 μl of each compound-pp mixture was added into each well. Examples of final concentrations of black seed oil and thymoquinone on the 384-well assay plate are set forth below in Table 25:

TABLE 25
An example of final concentrations of Temsavir
and Dolutegravir on the 384-well assay plate.

| | 1 | 2 | 3 | 4 | 5 | 6 | 7 | 8 | 9 | 10 | 11 | 12 | 13 | |
|---|---|---|---|---|---|---|---|---|---|---|---|---|---|---|
| A | 100 | 100 | 30 | 30 | 10 | 10 | 3 | 3 | 1 | 1 | 0.3 | 0.3 | 0.1 | Temsavir (μM) |
| B | 100 | 100 | 30 | 30 | 10 | 10 | 3 | 3 | 1 | 1 | 0.3 | 0.3 | 0.1 | Temsavir (μM) |
| C | 10 | 10 | 3 | 3 | 1 | 1 | 0.3 | 0.3 | 0.1 | 0.1 | 0.03 | 0.03 | 0.01 | Dolutegravir |
| | | | | | | | | | | | | | | (μM) |
| D | 10 | 10 | 3 | 3 | 1 | 1 | 0.3 | 0.3 | 0.1 | 0.1 | 0.03 | 0.03 | 0.01 | Dolutegravir |
| | | | | | | | | | | | | | | (μM) |
| E | 100 | 100 | 30 | 30 | 10 | 10 | 3 | 3 | 1 | 1 | 0.3 | 0.3 | 0.1 | Temsavir (μM) |
| | | | | | | | | | | | | | | with 0.08% |
| | | | | | | | | | | | | | | Black seed Oil |
| F | 100 | 100 | 30 | 30 | 10 | 10 | 3 | 3 | 1 | 1 | 0.3 | 0.3 | 0.1 | Temsavir (μM) |
| | | | | | | | | | | | | | | with 0.08% |
| | | | | | | | | | | | | | | Black seed Oil |
| G | 10 | 10 | 3 | 3 | 1 | 1 | 0.3 | 0.3 | 0.1 | 0.1 | 0.03 | 0.03 | 0.01 | Dolutegravir |
| | | | | | | | | | | | | | | (μM) with |
| | | | | | | | | | | | | | | 0.08% Black |
| | | | | | | | | | | | | | | seed Oil |
| H | 10 | 10 | 3 | 3 | 1 | 1 | 0.3 | 0.3 | 0.1 | 0.1 | 0.03 | 0.03 | 0.01 | Dolutegravir |
| | | | | | | | | | | | | | | (μM) with |
| | | | | | | | | | | | | | | 0.08% Black |
| | | | | | | | | | | | | | | seed Oil |
| I | 100 | 100 | 30 | 30 | 10 | 10 | 3 | 3 | 1 | 1 | 0.3 | 0.3 | 0.1 | Temsavir (μM) |
| | | | | | | | | | | | | | | with 1 μM |
| | | | | | | | | | | | | | | Thymoquinone |
| J | 100 | 100 | 30 | 30 | 10 | 10 | 3 | 3 | 1 | 1 | 0.3 | 0.3 | 0.1 | Temsavir (μM) |
| | | | | | | | | | | | | | | with 1 μM |
| | | | | | | | | | | | | | | Thymoquinone |
| K | 10 | 10 | 3 | 3 | 1 | 1 | 0.3 | 0.3 | 0.1 | 0.1 | 0.03 | 0.03 | 0.0 | Dolutegravir |
| | | | | | | | | | | | | | | (μM) with 1 μM |
| | | | | | | | | | | | | | | Thymoquinone |
| L | 10 | 10 | 3 | 3 | 1 | 1 | 0.3 | 0.3 | 0.1 | 0.1 | 0.03 | 0.03 | 0.01 | Dolutegravir |
| | | | | | | | | | | | | | | (μM) with 1 μM |
| | | | | | | | | | | | | | | Thymoquinone |

| | 14 | 15 | 16 | 17 | 18 | 19 | 20 | 21 | 22 | 23 | 24 |
|---|---|---|---|---|---|---|---|---|---|---|---|
| A | 0.1 | 0.03 | 0.03 | 0.01 | 0.01 | 0.003 | 0.003 | 0.001 | 0.001 | 0 | 0 |
| B | 0.1 | 0.03 | 0.03 | 0.01 | 0.01 | 0.003 | 0.003 | 0.001 | 0.001 | 0 | 0 |
| C | 0.01 | 0.003 | 0.003 | 0.001 | 0.001 | 0.0003 | 0.0003 | 0.0001 | 0.0001 | 0 | 0 |
| D | 0.01 | 0.003 | 0.003 | 0.001 | 0.001 | 0.0003 | 0.0003 | 0.0001 | 0.0001 | 0 | 0 |
| E | 0.1 | 0.03 | 0.03 | 0.01 | 0.01 | 0.003 | 0.003 | 0.001 | 0.001 | 0 | 0 |
| F | 0.1 | 0.03 | 0.03 | 0.01 | 0.01 | 0.003 | 0.003 | 0.001 | 0.001 | 0 | 0 |
| G | 0.01 | 0.003 | 0.003 | 0.001 | 0.001 | 0.0003 | 0.0003 | 0.0001 | 0.0001 | 0 | 0 |
| H | 0.01 | 0.003 | 0.003 | 0.001 | 0.001 | 0.0003 | 0.0003 | 0.0001 | 0.0001 | 0 | 0 |
| I | 0.1 | 0.03 | 0.03 | 0.01 | 0.01 | 0.003 | 0.003 | 0.001 | 0.001 | 0 | 0 |
| J | 0.1 | 0.03 | 0.03 | 0.01 | 0.01 | 0.003 | 0.003 | 0.001 | 0.001 | 0 | 0 |
| K | 0.01 | 0.003 | 0.003 | 0.001 | 0.001 | 0.0003 | 0.0003 | 0.0001 | 0.0001 | 0 | 0 |
| L | 0.01 | 0.003 | 0.003 | 0.001 | 0.001 | 0.0003 | 0.0003 | 0.0001 | 0.0001 | 0 | 0 |

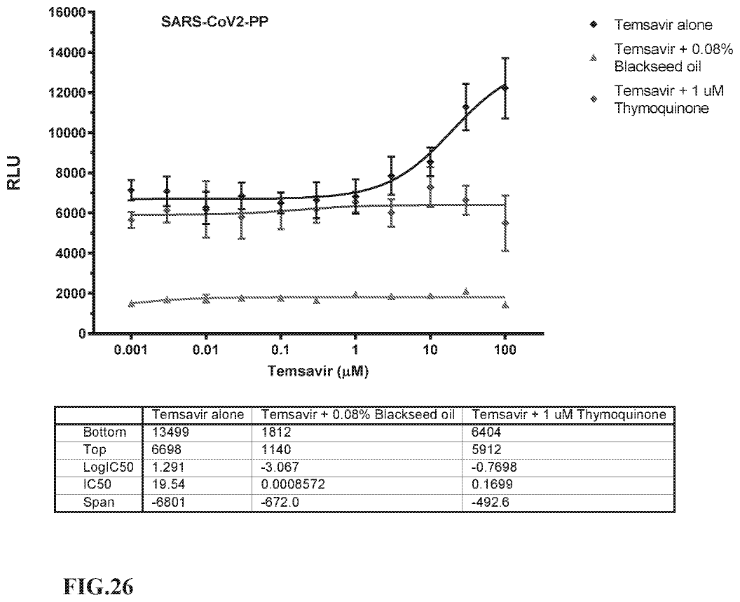

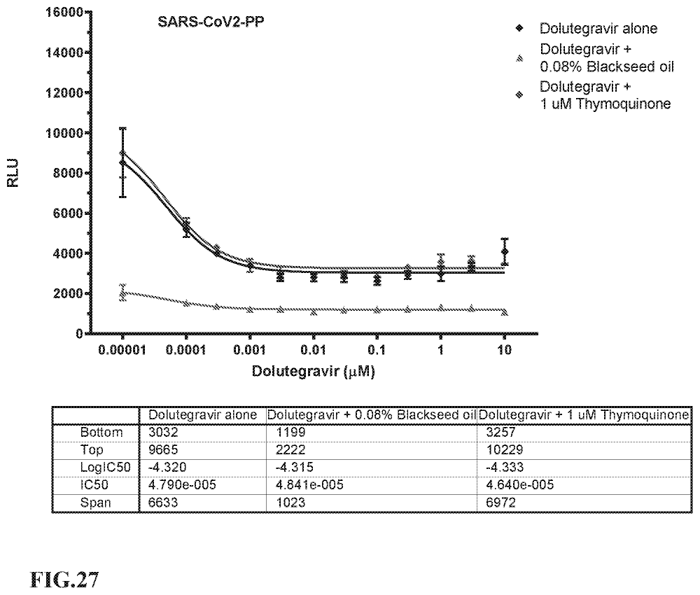

The plate was centrifuged at 54 g for 15 min at 4° C. and additional 7.5 μl of culture medium was then added into each well. The total final volume in each well was 25 μl. The cells were then incubated at 37° C. for 42 hr. Luciferase activities were measured with Firefly Luciferase Assay Kit (CB-80552-010, Codex BioSolutions Inc). IC₅₀ values were calculated based on curve fitting in GraphPad Prism. The results for temsavir are shown in FIG. 26, and the results for dolutegravir are shown in FIG. 27. In each graph, the x-axis depicts the temsavir or dolutegravir concentration, and the Y-axis depicts the relative luminescence unit (RLU), reflecting the luciferase activity and the viral infectivity.

These results indicate that temsavir does not inhibit SARS-CoV-2 MLV-PP infecting Expi-293F-ACE2 cells. To the contrary, temsavir may increase the infection of SARS-CoV-2 MLV-PP. Dolutegravir, on the other hand, is a potent inhibitor for SARS-CoV-2 MLV-PP infection. The addition of 1 μM of thymoquinone, and to an even greater extent 0.08% of black seed oil, further inhibits SARS-CoV-2 MLV-PP infecting Expi-293F-ACE2 cells.

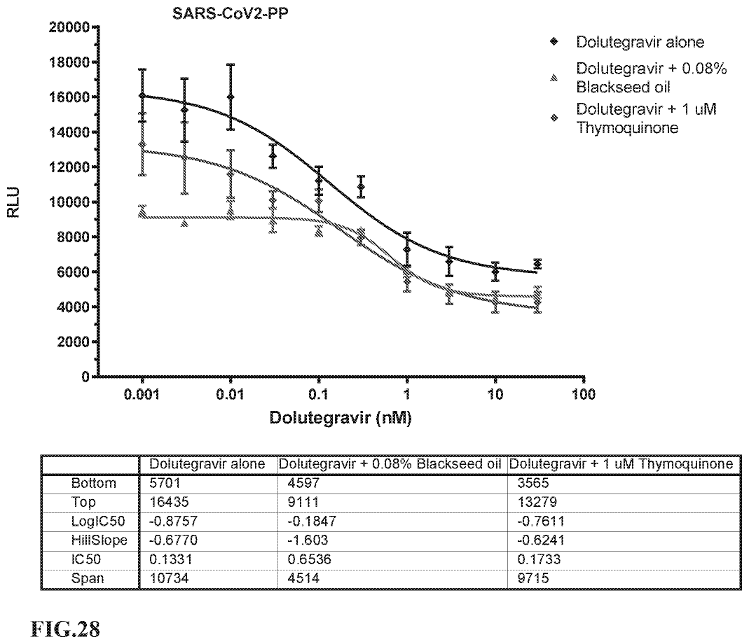

The dolutegravir experiment was repeated based on the above-described procedures, except that lower concentrations of dolutegravir were used. The results are shown in FIG. 28, and confirm that dolutegravir is a potent inhibitor of SARS-CoV-2 MLV-PP.

### Example 5: Randomized, Double-Blind, Placebo-Controlled Study to Evaluate the Safety and Efficacy of Black Seed Oil in Treating Participants Who have Tested Positive for COVID-19

A randomized (1:1), double-blind, placebo-controlled phase 2 study was conducted to assess safety and efficacy of black seed oil capsules versus placebo in treating patients who have tested positive for novel Coronavirus 2019 (COVID-19) in the outpatient setting. Inclusion criteria included: age 18 and over, presentation with recent mild to moderate clinical symptoms of COVID-19 infection, positive COVID-19 infection confirmed with a rapid antigen test at screening (or RT-PCR within the last 3 days) and confirmed with a RT-PCR test at baseline, and a score of on a minimum of 2 symptoms on the Modified FLU-PRO Plus.

Patients were treated with black seed oil at a dose of 500 mg, 3 capsules, two times a day for 14 days from date of randomization. Black seed oil was filled into enteric (acid-resistant) hard shell capsules, produced and tested under cGMP conditions. Quantitative viral load as measured by RT-PCR will be evaluated at baseline and on days 7 and 14. Covid-19 symptoms will be measured daily throughout the study until Day-14 using FLU-PRO Plus.

A primary objective of the study is to evaluate whether treatment with 3 g black seed oil (500 mg per capsule, 3 capsules BID) given orally on an outpatient basis can significantly reduce quantitative viral load by Day 7 compared to placebo in participants with COVID-19 infection, and/or can significantly reduce symptom burden in six domains measured by FLU-PRO Plus, namely, Nose, Throat, Eyes, Chest/Respiratory, Gastrointestinal, and Body/Systemic symptom burden by Day 7 from the start of therapy compared to placebo in participants with COVID-19 infection. The study will also compare the viral load profile overtime between treatment with 3 g Black Seed Oil (500 mg per capsule, 3 capsules BID) given orally on outpatient basis and placebo in participants with COVID-19 infection, and compare the percentage of RT-PCR negative (i.e., viral clearance) on Day 7 and Day 14 in participants taking 3 g Black Seed Oil (500 mg per capsule, 3 capsules BID) versus participants taking placebo in participants with COVID-19 infection. It is recommended that doses be taken with food, approximately 12 hours apart. Participants may continue with their normal standard of care, including any supplements or vitamins.

The study compared the duration and severity of symptoms measured by FLU-PRO Plus from Day 1 to Day 14 in total and sub-domain scores, namely, Nose, Throat, Eyes, Chest/Respiratory, Gastrointestinal, and Body/Systemic, in addition to taste/smell status of patients over time, between treatment with 3 g black seed oil (500 mg per capsule, 3 capsules BID) given orally on outpatient basis and placebo in participants with COVID-19 infection, and investigated if there exists an association between viral load and symptom severity by study arm and if such associations change overtime. The study also evaluated the safety and tolerability of Black Seed Oil 500 mg oral capsule, 3 capsules BID when given to participants with COVID-19 infection. Further, the study is in the process of evaluating the basic pharmacokinetics of black seed oil active ingredient (thymoquinone) at same time points (Days 1, 7, and 14) in participants with COVID-19 infection, and exploring the effect of black seed oil on inflammatory cytokines, coagulation factors and effector immune cells at same time points (Days 1, 7, and 14) in participants with COVID-19 infection.

The study included the measurement of change in quantitative viral load from baseline at Day 7 measured by RT-PCR in participants taking 3 g Black Seed Oil (500 mg per capsule, 3 capsules BID) versus participants taking placebo in participants with COVID-19 infection, and the measurement of symptom burden change from Day 1 to Day 7 in symptom subdomains nose, throat, eyes, chest/respiratory, gastrointestinal, and body/systemic measured by FLU-PRO Plus in participants taking 3 g Black Seed Oil (500 mg per capsule, 3 capsules BID) versus participants taking placebo in participants with COVID-19 infection.

The study also included measurement of change in quantitative viral load from baseline, Day 7, and Day 14 measured by RT-PCR in participants taking 3 g Black Seed Oil (500 mg per capsule, 3 capsules BID) versus participants taking placebo in participants with COVID-19 infection, and the percentage of negative RT-PCR (i.e., viral clearance) on Day-7 and Day-14 in participants taking 3 g Black Seed Oil (500 mg per capsule, 3 capsules BID) versus participants taking placebo in participants with COVID-19 infection.

The study also measured the severity and its change in Covid-19 symptoms as total score as well as sub-scores (Nose, Throat, Eyes, Chest/Respiratory, Gastrointestinal, and Body/Systemic symptom) in addition to the taste and smell status measured through FLU-PRO Plus from each day while on study therapy from Day-1 through Day-14 in participants with COVID-19 infection treated either with 3 g Black Seed Oil (500 mg per capsule, 3 capsules BID) or placebo. The correlation coefficient of quantitative viral load and symptom severity at baseline, at Day-7, and Day-14 in patients with COVID-19 infection was determined. Also, the number of adverse reactions reported in participants taking 3 g Black Seed Oil (500 mg per capsule, 3 capsules BID) versus participants taking placebo in participants with COVID-19 infection. All adverse events and serious adverse events was captured throughout the study as per schedule of assessments.

The study also included measurement of thymoquinone concentration in the plasma on Day 1, Day 7 and 14 using HPLC in patients treated with Black Seed Oil, and measurement of the inflammatory cytokine production, coagulation factors and the various effector immune cell subsets in the PBMC of these patients on Day 1, Day 7 and 14 using FACS.

Three cohorts of patients (n=50, 51 and 42 patients in the analysis pools of each respective cohort) were randomized 1:1 to receive either Black Seed Oil Capsules+Standard of Care (SOC) or placebo+SOC. After obtaining informed consent and meeting inclusion criteria, baseline assessments were performed at day 1 and the initial study intervention began. Follow-up assessments of study endpoints and safety occurred at days 4, 7, 10 and 14. Final assessments occurred at day 21—the final study visit. A follow up phone call occurred later.

Results

In summary, the Black Seed Oil Capsules were found to be safe and tolerable and led to significantly faster decline in total symptom burden (p<0.01), defined as duration and severity of symptoms (measured by Modified FLU-PRO Plus) over time from Day 1 through Day 14 in total FLU-PRO Plus symptom severity score overall and in sub-domain scores (namely, Throat, Gastrointestinal, Body/Systemic), between treatment with 3 g TQ Formula (500 mg per capsule, 3 capsules BID) given orally on outpatient basis and placebo in participants with COVID-19 infection. The study indicates that the black seed oil capsules were safe; less AEs reported with it than the placebo. Three out of 29 (10.3%) patients treated with TQ formula experienced a total of 3 treatment related AEs (2 mild, 1 moderate) while 6 out of 23 (26.1%) patients treated with placebo experienced a total of 9 treatment related AEs (8 mild, 1 moderate) (p=0.16).

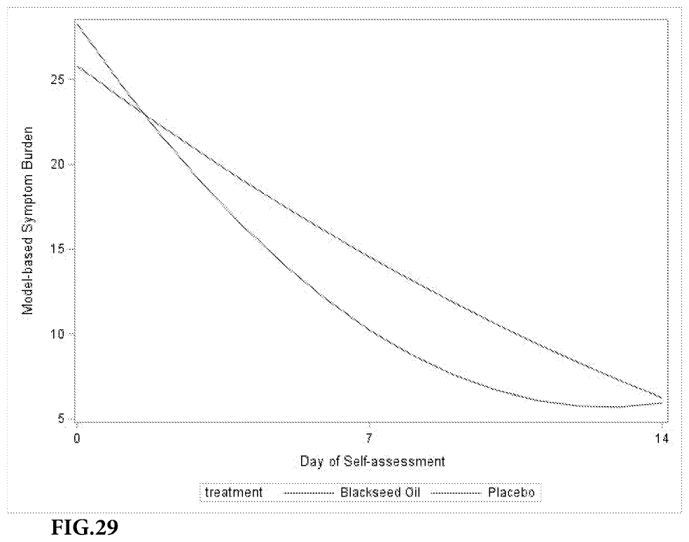

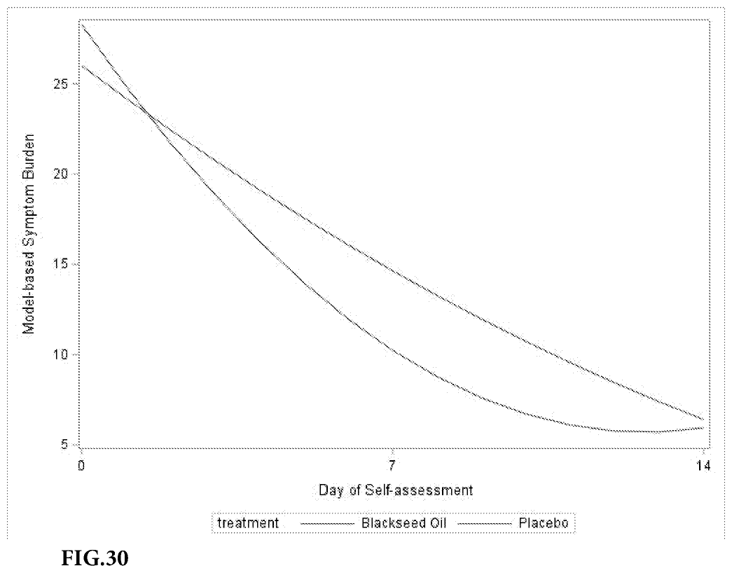

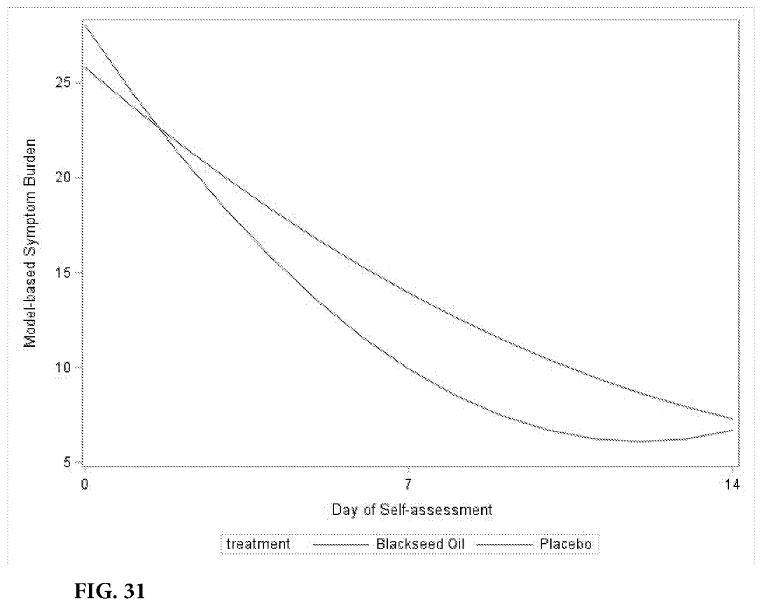

The duration and severity of symptoms over time from day 1 to day 14 was analyzed based on patient-reported total FLU-PRO Plus symptom severity score, both overall and in sub-domains scores (nose, throat, eyes, chest/respiratory, gastrointestinal, body/systemic and taste/smell). To determine total symptom burden the symptom score was added for each subject, and compared using Random Coefficients Models. In each of the three cohorts, the change in the total symptom burden in the Blackseed arm is more rapid (i.e., more rapidly decreasing) following a quadratic behavior, while the change in the placebo arm is more linear. This is shown in FIG. 29 (first cohort), FIG. 30 (second cohort) and FIG. 31 (third cohort). Quadratic vs. linear (blackseed arm vs. placebo) behavior in the modeled sub-domain scores amongst all cohorts for throat, gastro-intestinal, and body/systemic subdomains. These results indicate a statistically significant reduction in patient reported symptoms in this double blinded study.

Sustained clinical response was also analyzed. Sustained clinical response for purposes of this study is defined as a reduction of scores to less than or equal to 2 on all symptoms of the Modified FLU-PRO Plus. In two of the three cohorts, the blackseed oil group achieved a higher percentage of clinical response and higher percentage of sustained clinical response, both as compared to placebo. In the third cohort, the blackseed oil and placebos showed similar percentages of clinical response and sustained clinical response. Of the responders in each of the three cohorts, the median time to sustained clinical response was 6 days in the blackseed oil group and 8 days in the placebo group, though this difference did not meet statistical significance. Also, at day 14, the blackseed arm in each of the three cohorts had lower RT-PCR positivity rates (more favorable covid-19 recovery), though this trend did not reach statistical significance.

The viral load analysis was complicated by a maximum 25,000 viral load detection level, in which viral loads exceeding 25,000 were necessarily taken to be 25,000 for purposes of analysis. In each of the three cohorts, however, the blackseed oil arm had a lower median and mean viral loads at day 14, though the difference did not reach statistical significance under the constraints of this study.

The original scale of the viral load from the RT-PCR testing is copies/μ1 and any value greater than 25000 copies per μ1 is reported as ‘>25.000’, which was expressed in the protocol specified data analyses as 25,000, conservatively. If the RT-PCR test turns out to be negative, viral load measure is not provided and assumed zero, which was expressed as zero as well in the data analyses. Based on this original scale of viral load, cross-sectionally, there was no difference between the two arms of the study, namely, Blackseed Oil and Placebo arms, at baseline and on Day-7 of the assessment; however, there was a suggestive difference between the two arms in the original scale (the mean viral load of 4488 and 9559 respectively for Blackseed and Placebo arms, p=0.17), and in the log-scale (means of 2.43 and 4.37 respectively for Blackseed and Placebo arms, p=0.17).

An alternative analysis was also conducted to address the accumulation of the viral load data at 25,000 by projecting these cases to much higher viral load values using the close-to-perfect correlation between Viral Load and Cycle Threshold (CT) values provided concurrently from these RT-PCR assessments. Based on this CT-guided projection version as well, the above suggestive results remained valid with the log-scale means of 2.59 and 4.63 respectively for Blackseed and Placebo arms, p=0.20. The longitudinal models suggested time*treatment arm interaction suggesting that the viral load distribution was declining faster in patients treated with Blackseed Oil compared to those treated with Placebo (p=0.18).

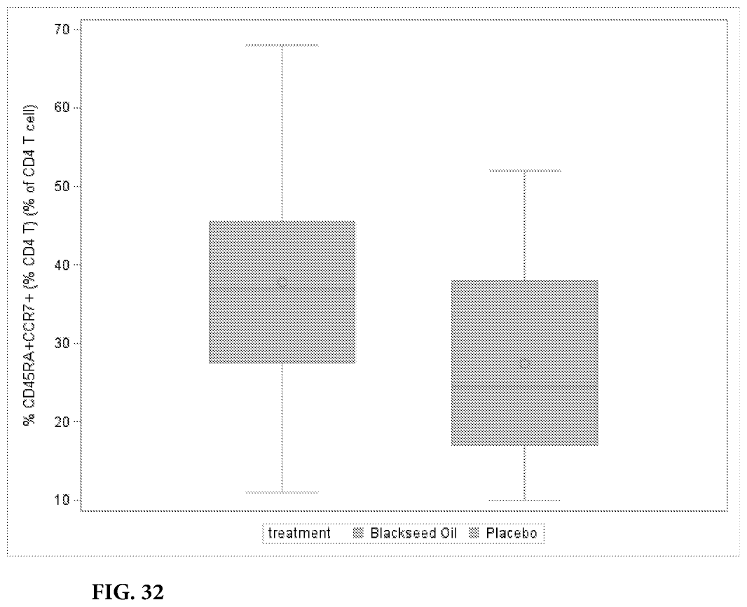

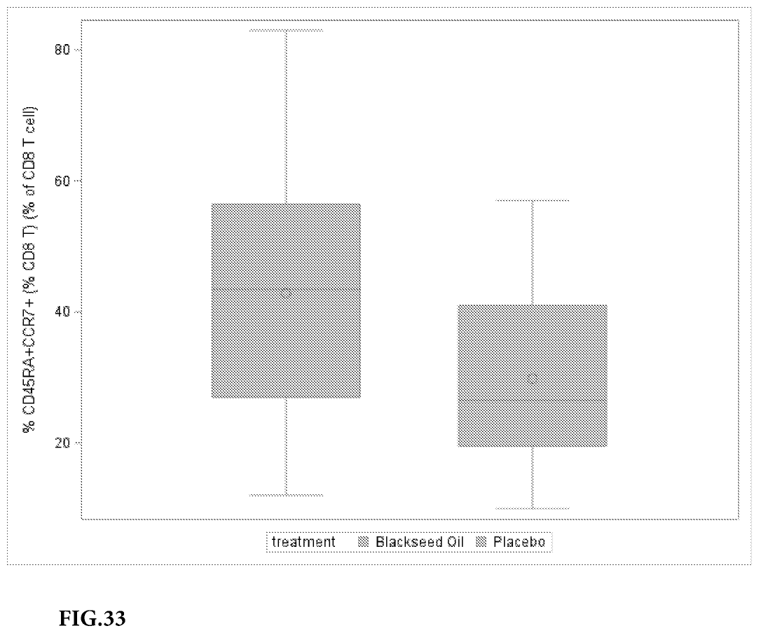

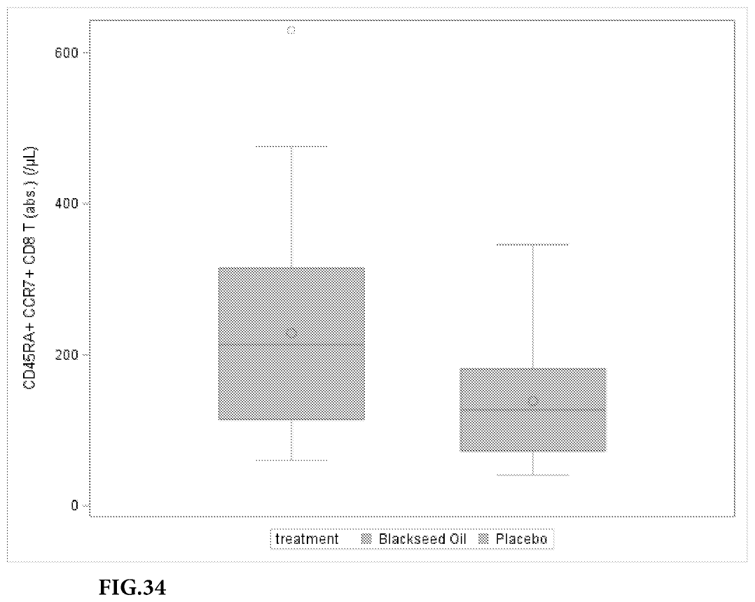

The effects of the black seed formulation on inflammatory cytokines, coagulation factors and effector immune cells at days 1 (baseline), 7 and 14 was explored in trial subjects. The blackseed arm did not demonstrate an increased immune response generally at day 7, nor did a comparison of Log of CD+4+CD8+T(abs.)(/uL) at day 14 demonstrate an increased blackseed oil response. Notably, however, the % CD45RA+CCR7+(% CD4T)(% of CD4 Tcell), the % CD45RA+CCR7+(% CD8 T)(% of CD8 T cell) and the CD45RA+CCR7+CD8T(abs.)(/μL) at day 14 were all higher in the blackseed treatment arms across all cohorts analyzed to date (analysis ongoing). Representative results for Cohort 1 are set forth in: FIG. 32, which shows a comparison of % CD45RA+CCR7+(% CD4T)(% of CD4 Tcell) between the treatment arms on day 14, the blackseed oil arm shown on the right; FIG. 33, which shows a comparison of % CD45RA+CCR7+(% CD8 T)(% of CD8 T cell), the blackseed oil arm shown on the right; and FIG. 34, which shows a comparison of CD45RA+CCR7+CD8T(abs.)(/μL), the blackseed oil arm shown on the right.

In COVID patients, clinical and laboratory parameters correlated with increased levels of proinflammatory cytokines in the acute phase, and subsequently evocative of a cytokine storm that strongly reminiscent of cytokine release syndrome (CRS) and a hall-mark of severe COVID-19. Monocytes and macrophages play a key role in pathological inflammation in COVID patients. Thymoquinone, the major component of black seed oil, has been shown to downregulate proinflammatory cytokines, modulate the production of type I IFN and TNF-α in monocytes and macrophages. Thus, both the antiviral and immunomodulatory activity of black seed oil could contribute to the positive outcome of this clinical trial.

In addition to pronounced systemic inflammation in severe COVID-19, lymphopenia is another prominent markers of COVID-19 and has been observed in over 80% of patients. The absolute numbers of CD4⁺ T Cells, CD8⁺ T Cells and B Cells were all gradually decreased with increased severity of illness. T Cells exhibit elevated exhaustion levels and reduce functional diversity. As shown in FIGS. 32-34, we observe significantly increased CD4⁺ and CD8⁺ T Cells with native/central memory phenotype (CD45RA⁺CCR7⁺), 14 days post-treatment compared to placebo. The data could be indicative of the immune recovery of black seed oil treated patients. It is also possible that the treatment might directly prevent overall T Cell exhaustion and promote SAR-CoV-2-specific T Cell proliferation.

### Example 6: Oral Black Seed Oil Formulation

Emulsions of the composition set forth below can be prepared by standard techniques, processed under an inert atmosphere at a temperature of about 5° C. and filled into capsules suitable for human administration.

| | |
|---|---|
| Black seed oil (CAS Number 8014-13-9, Sigma Aldrich) | 54.7% |
| Water | 35% |
| Xylitol | 5% |
| Potassium sorbate | 0.1% |
| Grindsted FF 1125 (Danisco A/S) | 4% |
| Lemon Flavor | 0.6% |
| Rosemary extract | 0.2% |
| Citric acid | 0.2% |
| Orange flavor | 0.1% |
| Antioxidant | 0.1% |
| Total | 100% |

All publications, patent and patent applications mentioned in this specification are incorporated herein by reference to the same extent as if each individual publication, patent or patent application was specifically and individually incorporated by reference.

While this invention has been particularly shown and described with references to preferred embodiments thereof, it will be understood by those skilled in the art that various changes in form and details can be made therein without departing from the scope of the invention encompassed by the appended numbered embodiments. Further, all embodiments included herein are given solely for the purpose of illustration and are not to be construed as limitations of the present invention, as many variations thereof are possible without departing from the spirit and scope of the invention.

1. A method of preventing or treating COVID-19 infection in a subject comprising administering to the subject an effective amount of thymoquinone.

2. The method of claim 1, wherein the subject is administered a composition that includes black seed oil.

3. The method of claim 1, wherein the thymoquinone is administered prophylactically prior to a diagnosed infection to prevent COVID-19 infection.

4. The method of claim 2, wherein the black seed oil is administered prophylactically prior to a diagnosed infection to prevent COVID-19 infection.

5. The method claim 1, wherein the thymoquinone is administered subsequent to a diagnosed infection to treat COVID-19.

6. The method of claim 2, wherein the black seed oil is administered subsequent to a diagnosed infection to treat COVID-19.

7. The method of claim 2 wherein the subject is administered black seed oil and an additional amount of thymoquinone, in combination.

8. The method of claim 1, further comprising administering an antiretroviral medication, in combination.

9. The method of claim 6, wherein the antiretroviral medication includes dolutegravir, or a pharmaceutically acceptable salt thereof.

10. The method of claim 1, further comprising administering a fatty acid.

11. The method of claim 10, wherein the fatty acid is selected from the group consisting of oleic acid, linoleic acid and palmitic acid, and pharmaceutically acceptable salts of the foregoing.

12. The method of claim 2, wherein the composition that includes black seed oil includes at least 1.6 wt % thymoquinone.

13. The method of claim 12, wherein the composition that includes black seed oil includes at least 2.0 wt % thymoquinone.
